(19) United States
(12) Patent Application Publication
(10) Pub. No.: US 2020/0115762 A1
(43) Pub. Date: Apr. 16, 2020

(54) BIOLOGICAL STATUS DETERMINATION USING CELL-FREE NUCLEIC ACIDS

(71) Applicant: Regents of the University of Minnesota, Minneapolis, MN (US)

(72) Inventors: Ali Khammanivong, Roseville, MN (US); Kelly Makielski, Minneapolis, MN (US); Jaime F. Modiano, Minneapolis, MN (US); Jong Hyuk Kim, Shoreview, MN (US); Milcah C. Scott, Minneapolis, MN (US); Alicia Donnelly, Malvern, PA (US); Hirotaka Tomiyasu, Tokyo (JP)

(21) Appl. No.: 16/600,486

(22) Filed: Oct. 12, 2019

Related U.S. Application Data
(60) Provisional application No. 62/745,129, filed on Oct. 12, 2018.

(51) Int. Cl. C12Q 1/6886; G16H 50/30; G16B 40/10; G16H 50/20; G16H 50/70; G16H 50/50

(52) U.S. Cl. CPC C12Q 1/6886; G16H 50/30; G16H 50/50; G16H 50/20; G16H 50/70; G16B 40/10

(57) ABSTRACT

The techniques and systems described herein relate to using machine learning models to associate a known biological state of an organism with patterns of expression exhibited by the organism of genes of a gene signature associated with a disease state, such as to train the machine learning models to determine unknown biological states associated with the patterns of expression. Some techniques include determining an unknown biological status of an organism based on an expression pattern of genes of a gene signature in the organism, which the machine learning model may compare to known expression patients learned during the training technique. The expression patterns may be determined based on sequences of exosomal RNAs isolated from exosomes from a sample of bodily fluid from the organism and an approximate number of times each RNA sequence that substantially aligns with a gene of the gene signature occurs in the sample of bodily fluid.

This disclosure claims the benefit of U.S. Provisional Patent Application No. 62/745,129, entitled “BIOLOGICAL STATUS DETERMINATION USING CELL-FREE NUCLEIC ACIDS” and filed on Oct. 12, 2018, the entire contents of which are incorporated herein by reference.

### TECHNICAL FIELD

This disclosure relates generally to methods for determining a biological status of an organism and, more specifically, methods for determining a biological status of an organism based on the identification and analysis of cell-free nucleic-acid biomarkers.

### BACKGROUND

The composition and abundance of an organism's nucleic acids provide biomarkers indicative of various aspects of the organism's genome and transcriptional expression, including the organism's predisposition toward particular biological status, as well as the presence and progression of such biological status. Much of a living, multicellular organism's total nucleic acid complement is located intracellularly: DNA is chiefly located within the nuclei of the cells, whereas RNA of numerous types is abundant within the various organelles and cytoplasm of cells. Nucleic acids derived from cells may be used as biomarkers to determine a biological status of organism, such as a predisposition toward development of a disease, the presence of the disease, or the biological behavior of the disease.

### SUMMARY

This disclosure describes example techniques and systems for determining a biological status of an organism based on the abundance of nucleic acids associated with genes of a gene signature in the blood of the organism, such as genes from exosomes present in blood of the organism. A gene signature (e.g. art exosomal gene signature) may be a plurality of genes associated with at least one biological status, such as a presence or absence of a disease state, a likelihood of development of a disease state, one or more characteristics of an existing disease state, a likelihood of a future progression of an existing disease state, one or more characteristics of a predicted future progression of an existing disease state, or a probability that an organism may respond to a specific therapy. Techniques described herein may include using one or more machine learning models to determine different patterns of gene expression of a plurality of gene sequences of a gene signature that are associated with different biological statuses corresponding to a particular disease state, such as based on samples from organisms having known biological statuses. Gene signatures from exosomes will be described as examples herein, but similar techniques may be applied to serum (e.g., cell-free RNA) or tissue samples. Such different patterns of gene expression then may be used by the one or more machine learning models to determine a previously-unknown biological status of an organisms based on a pattern of gene expression of the plurality of gene sequences of the gene signature exhibited by the organism. In one example, a gene signature that has been identified as being associated with biological statuses corresponding to osteosarcoma (OS or OSA) in dogs includes the following canine genes: SKA2, NEU1, PAF1, PSMG2, and NOB1. As discussed below, the relative expression levels of these genes may vary between dogs having different biological statuses corresponding to OS, and may be used in techniques for determining a biological status of an organism (e.g., a dog).

In one example, a method for screening an organism for osteosarcoma comprises isolating a plurality of exosomes from a sample of bodily fluid derived from the organism, wherein the plurality of exosomes comprises a plurality of molecules of ribonucleic acid (RNA); determining respective RNA sequences for the plurality of molecules of RNA; analyzing, by processing circuitry and using one or more machine teaming models, expression level exhibited by the organism of each of a plurality of genes of a gene signature of osteosarcoma-linked genes based on the RNA sequences of the plurality of molecules of RNA occurring in the sample; and determining, based on the analysis, a biological status of the organism.

In another example, a method comprises obtaining a plurality of exosomes from each of a plurality of samples of bodily fluid derived from corresponding ones of a plurality of subjects (e.g., individual organisms of a same species), wherein one or more first subjects of the plurality of subjects have a biological status different from a biological status of one or more second subjects of the plurality of subjects, wherein the plurality of exosomes from each of the plurality of samples of bodily fluid comprises a plurality of molecules of ribonucleic acid (RNA); for each of the plurality of samples of bodily fluid: determining, for substantially each molecule of the plurality of molecules of RNA, a corresponding RNA sequence; determining, for each corresponding RNA sequence, whether the RNA sequence is associated with exactly one corresponding gene sequence of a gene signature comprising a plurality of gene sequences; determining an approximate number of times that each RNA sequence associated with exactly one corresponding gene of the gene signature occurs in the sample of bodily fluid; and determining, using one or more machine learning models, a pattern of expression of the plurality of gene sequences of the gene signature associated with the sample of bodily fluid based on the approximate number of times that each RNA sequence substantially aligned with exactly one corresponding gene of the gene signature occurs in the sample of bodily fluid; and associating, using the one or more machine learning models and for each subject of the plurality of subjects, the biological status of the subject with the corresponding pattern of expression of the of the plurality of gene sequences of the gene signature associated with the sample of bodily fluid from the subject.

In another example, a method comprises obtaining a plurality of exosomes from a sample of bodily fluid derived from an organism, wherein the plurality of exosomes comprises a plurality of molecules of RNA; determining, for substantially each molecule of the plurality of molecules of RNA, a corresponding RNA sequence; determining, for each corresponding RNA sequence, whether the RNA sequence is associated with exactly one corresponding gene sequence of a gene signature comprising a plurality of gene sequences; determining an approximate number of times that each RNA sequence substantially aligned with exactly one corresponding gene of the gene signature occurs in the sample of bodily fluid; analyzing, using one or more machine learning models, the approximate number of times that each RNA sequence associated with exactly one corresponding gene of the gene signature occurs in the sample of bodily fluid; determining, using one or more machine learning models and based on the analysis, a pattern of expression exhibited by the organism of each the plurality of genes of the gene signature; comparing, using one or more machine learning models, the pattern of gene expression to at least one known pattern of gene expression, wherein each of the at least one known patterns of gene expression is associated with a biological status; and determining a biological status of the organism based on the comparison,

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a flow diagram illustrating an example technique for training a machine learning model in accordance with the examples of this disclosure.

FIG. 2 is a flow diagram illustrating an example technique for determining a biological status of an organism in accordance with the examples of this disclosure.

FIG. 3 is flow diagram illustrating an example technique for orthotopic xenograft model establishment, candidate biomarker gene discovery, and in-species validation of a gene signature that includes biomarker genes in accordance with the examples of this disclosure.

FIG. 4 is functional block diagram illustrating an example configuration of a computing system that may be used to implement the machine learning model training and biological status determination described herein.

FIG. 5 is a functional block diagram illustrating an example configuration of a computing device of the computing system of FIG. 4 that may be used to implement the machine learning model training and biological status determination described herein.

FIG. 6A includes digital images from exosome specific staining in human osteosarcoma tissues.

FIGS. 6B and 6C are graphs of expression levels for different markers across different stages of osteosarcoma.

FIG. 6D is digital images of exosomes isolated from different osteosarcoma (OS) cell lines in accordance with examples of this disclosure.

FIGS. 7A and 7B are scanning electron microscope (SEM) images, at different magnification levels, of representative exosomes isolated from OS cell lines in accordance with examples of this disclosure.

FIG. 7C is digital images of results of a Western blot assay illustrating quantities of three expression products isolated from each of five different cell lines in accordance with examples of this disclosure.

FIGS. 7D and 7E are graphical representations of exosome size and exosome quantification of exosomes isolated from different OS cell lines in accordance with examples of this disclosure.

FIGS. 8A-8D are graphical representations of exosome size and exosome quantification of exosomes isolated from different OS cell lines in accordance with examples of this disclosure.

FIGS. 8E-8F are graphical representations of exosome size and exosome quantification of example exosomes isolated from serum of a normal dog and from serum of a dog with osteosarcoma.

FIG. 9 is digital images of results of a Western blot assay illustrating presence and quantity of an exosomal protein CD63 and protein β-actin from whole-cell lysate and exosome isolates derived from each of four different OS cell lines in accordance with examples of this disclosure.

FIG. 10A is a digital image of OS cells transfected with a CD81-GFP fusion construct synthesizing and secreting green fluorescent protein (GFP)-positive tumor exosomes (TEX) in accordance with examples of this disclosure.

FIGS. 10B and 10C are brightfield and blue-light images of fibroblast and endothelial cells that had taken-up GFP-positive TEX produced by transfected OS cells of FIG. 10A at different times post-TEX introduction in accordance with examples of this disclosure.

FIGS. 10D and 10E are graphical representations of percentages of endothelial cells and fibroblasts, respectively, that had taken up GFP-positive TEX from the transfected OS cells of FIG. 10A versus time in accordance with examples of this disclosure.

FIGS. 10F and 10G are graphical representations of migration distance and cell count of different cell lines that had taken up GFP-positive TEX in accordance with examples of this disclosure.

FIG. 10H is a graphical representation of example results from a microarray-based analysis to quantify differential gene expression of human fibroblast cells treated with exosomes from OSCA 32 and OSCA 40 cell lines as compared to untreated human fibroblasts.

FIGS. 11A and 11B are digital images of results of gel electrophoresis assays illustrating; packaging of RNAs from genes transfected or engineered into OS cell lines into exosomes by OS cells in accordance with examples of this disclosure.

FIGS. 12A and 12B are photographic representations of data pertaining to the application of the techniques described herein with respect to the OS-1/OS-2 xenograft example.

FIG. 13A is a graphical representation of the detection of biomarkers of disease and host response in the OS-1/OS-2 xenograft example.

FIG. 13B is a graphical representation of B-H p-values for ingenuity pathway analysis (IPA) canonical pathways associated with functions of one or more of the 38 statistically significant differentially expressed genes (SEGS) illustrated in FIG. 13A in the OS-1/OS-2 xenograft example.

FIG. 14A is a graphical representation of a data analysis technique in accordance with the examples of this disclosure, as applied to the OS-1/OS-2 xenograft example.

FIG. 14B is a graphical representation of a data analysis technique in accordance with the examples of this disclosure, as applied to the OS-1/OS-2 xenograft example, indicating biological processes and canonical pathways associated with identified gene clusters.

FIG. 15 is a flow chart illustrating an example technique for selecting candidate biomarkers for in-species validation in accordance with the examples of this disclosure.

FIG. 16A is a graphical representation of differentially-expressed and co-regulated genes expressed by osteosarcoma that can be used as one or more biomarkers for osteosarcoma.

FIG. 16B is a graphical representation of the co-regulated genes of FIG. 16A and the process of in-species validation of those exosomes to detect the condition of interest in accordance with examples of this disclosure.

FIGS. 17A-17E are biomarker expression profiles of messenger RNA (mRNA) associated with each of five genes of an identified gene signature for each of a plurality of samples derived from subjects having different biological statuses.

FIG. 18A is a graphical representation of results of principal component analysis of the plurality of samples of FIGS. 17A-17E.

FIG. 18B is a graphical representation of results of linear discriminant analysis (LDA) of the plurality of samples of FIGS. 17A-17E.

FIG. 18C is a graph of example averaged machine learning performance comparison across four selected models.

FIG. 19 is probability plot of results of predicted classifications of a set of pre-treatment samples using a CN2 rule inducer model.

FIG. 20 is a chart illustrating classification accuracy of a set of samples based on cross-validation analysis with different machine learning methods.

FIGS. 21A and 21B are graphical representations of example survival probability of subjects classified using the machine learning models.

### DETAILED DESCRIPTION

In general, this disclosure describes example techniques related to determining a biological status of an organism based on a comparison of the organism's expression pattern of genes of a gene signature to known expression patterns of the genes of the gene signature that are associated with known biological statuses. These genes may be contained in exosomes such that the gene signature may be referred to as an exosomal gene signature or gene signature contained in exosomes in some examples as described herein. The gene signature may be associated with a particular disease state, such as a type of cancer. For example, when a subject has a disease such as cancer, the subject may normally gain or lose expression of a single gene over time. Therefore, looking for expression of a single gene associated with a disease may cause a false negatives or false positives due to normal variation in expression of that single gene. However, during a disease state, several genes may be expressed, or not expressed, coordinately together as an indication of that particular disease. As described herein, identifying a gene signature that incorporates several coordinately regulated (e.g., turned on or off together) genes may provide a more robust diagnosis of the subject because the gene signature of a plurality of genes being expressed may be influenced less by the occasional gene that may or may not be expressed when the subject is tested.

Biological statuses associated with the gene signature may indicate a presence or absence of the particular disease state in the organism, a likelihood that the organism may develop the disease state, one or more characteristics of the disease state in examples in which the disease state already exists in the organism (e.g., a disease stage or a likelihood of disease progression), one or more characteristics of a predicted future progression of an existing disease state, or a probability that the organism will respond to a particular therapy. As used in the description of the example techniques and systems described herein, a “disease state” may be associated with a particular disease (e.g., a particular type of cancer) or other physiological condition, or a type of disease (e.g., cancer in general) or type of physiological condition. In some examples, a physiological condition may be associated with biological status(es) such as a probability of response to therapy, with actual response to therapy, with probability of rejection of a transplantation, with actual rejection of an existing transplantation, or other responses of interest. Such techniques for determining a biological status of art organism may include using machine learning models to analyze sequences of RNA molecules present in a sample derived from the organism to determine the organism's expression pattern of genes of a gene signature and compare the organism's expression pattern to known expression patterns of the genes of the gene signature to determine a biological status of the organism.

This disclosure also describes example techniques relating to training such machine learning models to associate expression patterns of the genes of the gene signature from model organisms having different known biological statuses of a disease state or other physiological state associated with the gene signature. In one example, an exosomal gene signature that has been identified as being associated with biological statuses corresponding to osteosarcoma (OS or OSA) in dogs includes the following canine genes: SKA2, NEU1, PAF1, PSMG2, and NOB1. As discussed below, the relative expression levels of these genes may vary between dogs having different biological statuses corresponding to osteosarcoma This relative expression may refer to genes that are expressed in cells as mRNA transcripts that are loaded into exosomes. In this manner, identification of gene expression described herein may refer to quantifying the steady state number of transcripts mRNA transcripts) that are loaded in the exosome. The level of expression of a gene described herein may therefore be determined by quantifying the relative number of mRNA transcripts detected. The gene expression patterns of genes of the gene signature of the model organisms having known biological statuses then may be used as known expression patterns when determining an unknown biological status of a test organism.

In some examples, the techniques for determining a biological status of an organism and the techniques for training one or more machine learning models are illustrated using osteosarcoma as a disease state of interest and dogs as organisms of interest. However, the description herein of such techniques is not intended to be limiting. Such techniques may be applied to other disease states of interest or other physiological states of interest. Additionally, or alternatively, such techniques may be applied to other organisms of interest humans or other non-human animals).

Also described herein are techniques for identifying genes of a gene signature associated with a particular disease state or other physiological state for use in the techniques described herein. The techniques for identifying genes of a gene signature illustrated using orthotopic xenografts (i.e., tissue donor organisms) of canine osteosarcoma in nude mice host organisms). Such techniques may be applied to other disease states of interest or other physiological states of interest. Additionally, or alternatively, such techniques may be applied to other donor and/or host organisms.

Osteosarcoma (primary bone cancer) is a rare disease with a disproportionate impact in humans, as it mainly affects children, adolescents, and young adults. More than half of patients with osteosarcoma relapse and die from metastatic disease within 10 years of their initial diagnosis, highlighting the need for predictive biomarkers to personalize therapies. Osteosarcoma also among the most common tumors affecting dogs. Some transcriptional programs that predict tumor biological behavior, including metastasis, and thus inform prognosis for osteosarcoma patients (i.e., human or non-human animal patients) at the time of diagnosis through the use of innovative, multi-species comparative approaches. The most robust among these methods, called the Gene Cluster Expression Summary Score (GCESS), requires invasive tissue biopsies, and so it has not yet been widely adopted in practice also, its utility to monitor minimal residual disease is unknown. Thus, non-invasive tests that inform both prognosis and longitudinal remission status may be advantageous to aid in diagnosis and treatment of osteosarcoma patients.

Precision diagnostic techniques may help enable more efficient and cost-effective care for osteosarcoma patients. The drive for personalized medicine in cancer is being fueled by increasingly sophisticated understanding and classification of diseases that are paired with appropriate therapies, as well as by expanding pharmacogenomics. In cancer, the success of personalized medicine hinges on pairing the right therapy with the right patient and disease. In order to do this, reliable companion diagnostics that accurately predict cancer risk and prognosticate cancer progression may be advantageous. The example techniques described herein may identify biomarkers that can predict osteosarcoma behavior so that the type and intensity of therapy for individual patients can be tailored accordingly.

The behavior of human and canine osteosarcoma tumors can be determined by distinct gene expression profiles, which include a combination of tumor cell-intrinsic factors and tumor-microenvironment (TIME) extrinsic factors. Described herein is an example application of techniques for determining similar molecular profiles in canine spontaneous osteosarcoma using a minimally invasive platform for biomarker discovery based on detection of serum-derived exosomal gene transcripts and machine teaming. Some example techniques described herein may enable identification of biomarkers in serum exosomes. Since exosomes may be loaded by diseased cells and loaded by cells that respond to disease, exosomal gene transcripts (e.g., exosomal gene signatures) may enable to prediction of whether or not a subject likely has contracted, or will contract, a specific disease such as osteosarcoma. In some examples, the techniques described herein may enable identification of biomarkers that may predict osteosarcoma behavior and may enable stratification that minimizes risk and maximizes benefit through discovery of biomarkers in serum exosomes. Information obtained from such techniques may aid in development of new therapies for the highest risk patients. One advantage of serum biomarkers is that routine blood samples can be obtained using minimally invasive methods in patients where single or repeat biopsies are problematic, such as is true for osteosarcoma. One issue with serum exosomes is that the identification of relevant biomarkers requires isolating them from a background of molecules produced and secreted by trillions of cells. While this can be done statistically using big data approaches such as sequencing DNA, RNA, or proteins from exosomes in very large groups of patients with different patterns of disease behavior, it is costly, labor intensive, and time consuming.

The platform described herein may improve efficiency and/or lower cost, thereby mitigating the issue with using serum exosomes described above. For example, by using xenografts and next generation RNA sequencing, an environment where cargo in TEXs can be readily distinguished from cargo in host derived exosomes may be created. In the case of osteosarcoma, the tools needed to carry out such techniques are tumors that recapitulate the heterogeneity observed in patients. While patient-derived xenografts are one approach to achieve this heterogeneity, these are mostly implanted heterotopically, and in the case of osteosarcoma, heterotopic (subcutaneous) tumor implants do not recapitulate the behavior of the tumor. For example, some tumors may be recalcitrant to grow, others may show fibroblastic differentiation without production of osteoid matrix, and yet others may show aberrant patterns of metastasis. Osteosarcoma cell lines, on the other hand, show stable phenotypes and can be implanted orthotopically in long bones, recreating the normal tumor niche. Even the most recalcitrant osteosarcoma cell lines grow orthotopically in mice, and they retain the capability to metastasize to bones and lungs, at least in a few individual animals. Therefore, tumor heterogeneity may be recapitulated both within cell lines because metastatic efficiency may show some degree of variability, as well as among cell lines, because on average, they may show different propensity to metastasize. This behavior is independent of therapy and may be used to guide therapy, which may simplify the variables needed to create a reproducible model where disease progression is measured in days and the number of subjects needed is in the tens per group.

Described herein is an example method to identify species-specific mRNA sequences in exosomes from tumor xenografts (tumor, or donor species and stroma, or host species). This method may thus involve blood exosomes or serum exosomes, which may allow for discovery of biomarkers that may predict the presence of osteosarcoma in the donor organism. Also described herein are applications of techniques for in specie,s validation, which illustrate the capability of the trained machine-learning models described herein to accurately classify animals (in one example, dogs), such as into four biological status groups consisting of “healthy,” “osteosarcoma,” “other bone tumor,” or “other disease” using data from the xenograft model combined with machine learning algorithms.

The techniques described herein may enable identification of evolutionarily conserved features of disease (e.g., osteosarcoma), which may help enable development of novel diagnostic tests and treatment strategies. For example, a multi-species comparative approach is described herein as applied to laboratory animal models of osteosarcoma and spontaneous osteosarcoma in companion dogs, which develop this disease with much greater frequency than humans. Such techniques may enable identification of serum biomarkers that can be used to predict tumor behavior in osteosarcoma patients at the time of diagnosis.

In addition to the intrinsic advantages associated with developing minimally-invasive diagnostic tests, techniques for the successful validation of biomarkers as described herein may enable implementation of effective, patient-centered therapies, which may reduce the probability of treatment-related side effects in some examples, human osteosarcoma xenografts, canine osteosarcoma xenografts, and syngeneic models of mouse osteosarcoma with distinct metastatic propensities may be used as part of a platform for exosome biomarker discovery. Data obtained from such techniques may be used to generate conserved, exosome-associated mRNA signatures that may be associated with metastatic potential at diagnosis and/or with risk for relapse after treatment. Techniques described herein may validate exosome mRNA signatures identified in clinically annotated cohorts of samples from humans and/or dogs with osteosarcoma to establish their predictive value.

As described herein, xenograft models of osteosarcoma may enable discovery of exosome-based biomarkers that may be used to predict tumor biological behavior, and thus may inform prognosis of osteosarcoma patients. Such techniques may include validation of these biomarkers in well-annotated sample cohorts obtained from children and dogs with spontaneous osteosarcoma, which may provide a novel measure to assist pediatric oncologists in the management for this disease. Such techniques and results of example applications thereof are described below with respect to the following three purposes: (1) the techniques described herein may enable models of osteosarcoma with distinct metastatic propensity for exosome biomarker discovery; (2) the techniques described herein may generate conserved, exosome-associated mRNA signatures associated with metastatic propensity; and (3) the techniques described herein may validate exosome mRNA signatures in annotated cohorts of samples from children and dogs with osteosarcoma to establish their predictive value.

Orthotopic osteosarcoma xenograft models using human and canine cell lines that have distinct biological behavior and metastatic propensity may be created and metastasis in such models may be evaluated using in vivo imaging. In some such models, exosome uptake into the pulmonary microenvironment may be established in vivo using Cre-lox dual reporter mice. Exosomes may be isolated from cultured cells, and longitudinally from mouse serum samples, with emphasis on sample collection and exosome enrichment methods. mRNA cargo in exosomes may be characterized using next generation sequencing (NGS) and bioinformatics to identify evolutionarily conserved, exosome-associated mRNA clusters. mRNAs originating from tumor exosomes (TEX) and host exosomes may be identified. Potential alterations in exosomal mRNA content may be established for each parental cell line and its genetically modified derivatives. Analysis of the potential mRNA clusters may include unsupervised methods, as well as supervision by cell line and by outcome (time to metastasis). mRNA signatures may be assembled where each component meets criteria of detectable expression over background, low inter-sample variance, high inter-gene correlation, and cross-species conservation. A final list of mRNAs associated with a determined gene signature of osteosarcoma may be established based on the point of minimal returns across models, and linearity characteristics may be validated for each gene of the gene signature to meet rigorous criteria for quantification.

In some examples, archival serum samples from human and dog osteosarcoma patients may be obtained from the Children's Oncology Group (COG) and the Pfizer Canine Comparative Oncology and Genomics Consortium (CCOGC). Inc. Such samples have been collected and stored under rigorous standard protocols that ensure preservation of biological molecules. Preparation of exosomal RNA and quantification of transcript abundance (qRT-PCR and NanoString), including gene sequences used for calibration and normalization, may be done following FDA guidance. Outcome data may be blinded until all RNA data are collected and tabulated. Relationships between exosomal in:RNA signatures and patient outcomes may be analyzed using unsupervised methods, including principal components analysis (PCA) and supervised linear discriminant analysis (MA). Iterative training and validation may be used for machine learning algorithms. A probability that patients with more aggressive osteosarcoma (higher metastatic propensity) and with less aggressive osteosarcoma (lower metastatic propensity) are accurately classified by each algorithm may be determined. The common rate of success in the validation set may be used to define the operating true positive (sensitivity) and true negative (specificity) of the test using receiver operating characteristic (ROC) curves.

As described above, personalized medicine in diagnostic pathology may help address shortcomings of conventional practices of applying the same therapy across groups of people with one disease that shows heterogeneous biological behavior. Accurate, reproducible tests that can be readily translated into the clinical setting may help enable the application of current understandings of disease and the development of new therapies in practice. As noted above, more than half of human patients with osteosarcoma relapse and die from metastatic disease within 10 years of diagnosis. At present, there is a paucity of reliable tests to predict behaviors of osteosarcoma and/or to help enable individualization of therapy for osteosarcoma patients. While aggressive treatments may prevent or delay metastasis and achieve long-term survival, the intrinsic properties of the tumor seem to be major determinants of outcome, creating opportunities for personalized therapies. Specifically, therapy-related toxicity is a major concern in oncology. Thus, it may be advantageous to identify patients with a more favorable prognosis, such that these patients might be treated more conservatively, which may reduce the need for radical, disfiguring surgeries, diminishing the likelihood of cognitive deficits, and reducing the probability of secondary, treatment-related malignancies. Conversely, patients with worse prognoses could receive more aggressive treatments or be guided to experimental clinical trials that might improve their outlook for long-term survival.

Osteosarcoma also affects non-human animals, including dogs. The following example is an example of potential significance of the techniques described herein with respect to canine patients, but also may be applicable to human patients or other non-human animal patients. Some techniques described herein with respect to the example of osteosarcoma in dogs include quantification of the expression of a 6-gene signature associated with osteosarcoma plus a housekeeping control for normalization, and use of machine learning models or algorithms to establish the probability that a dog has osteosarcoma or a likelihood that the dog may develop osteosarcoma. This could be used to monitor dogs at high risk to identify the possible presence of osteosarcoma in advance of clinical signs, allowing for early intervention, as well as to monitor duration of remission or relapse. Quantification of the 5 genes may be used to create a “compound signature.” In this example, the compound signature includes 5 genes (normalized in expression level to the housekeeping gene). Any one gene individually, or groups of genes that include some, but not all the genes may not achieve the same effect.

Large and giant dogs (on average, mix breed dogs or purebred dogs weighing more than 20 kilograms) are at high risk of developing bone cancer. For some breeds, the lifetime risk is as high as 1 in 5 (20% for an individual), but there are no safe methods to diagnose the disease in its early stages (the most accurate method requires exposure to high levels of radiation through bone scan and carries additional risk and cost of anesthesia). Conventionally, there are no simple, low risk methods to monitor relapse in dogs that are receiving treatment. Instead, conventional, recommendations may include radiographs with multiple views every three months. By the time lesions are evident radiographically, any treatment or intervention has virtually no chance of success. CT scans appear to be more sensitive, but they still deliver high doses of radiation and carry additional risk of anesthesia. In contrast, the techniques described herein may be used to screen dogs at risk to establish the potential presence of osteosarcoma before the tumor creates clinical signs, which may reduce the number of dogs that would need to be exposed to bone scans (and justifying the risk of anesthesia and exposure for those individuals that test positive), and may help detect relapse early, which may allow for changes in the treatment strategy when they might still have a chance to be effective.

There are an estimated 80-90 million pet dogs in the US. Given breed and size distribution, more than 50% of pet dogs may carry high risk for osteosarcoma. On any given year, assuming a median age of 5 to 6 years of age for the population, more than 50% of dogs, or as many as 20-30 million, would comprise the “at risk” population that might benefit from this kind of test. That represents a lot of dogs and a lot of families in the US alone. While other tumors that arise within the bone and present like osteosarcoma in large dogs are rare (for example, hemangiosarcoma or malignant histiocytosis that arise in bone as the primary site), as are primary infections of bone (osteomyelitis), the techniques described herein may distinguish between primary bone cancer and other cancer types, as well as non-malignant conditions.

Conventionally, diagnosis of osteosarcoma is only done after clinical signs are evident. By then, more than 95% of dogs have micrometastatic disease, and 90% of these dogs may inevitably die from osteosarcoma regardless of treatment. There is no accepted or practiced method for early detection. The recommendation for evaluation of remission status is to do quarterly physical exams and radiographs. In addition to risks of radiation exposure and anesthesia, these tests are time consuming and costly, leading to reduced compliance. Conventionally, blood-based tests are not available. The blood-based test techniques described herein thus may reduce cost and risk, and may enhance convenience and compliance in osteosarcoma diagnosis and/or treatment.

A positive result of the test of the example techniques described herein, when used in the scenario of screening, may lead a clinician to order highly sensitive tests that can localize a tumor, such as a1-99 bone scan or PET-CT. Identification of a tumor early in its natural history could provide opportunities for treatment that preserve the limb (or the bone), and that are less aggressive and toxic than when a tumor is diagnosed in the advanced stages. Blood-based screening tests based on the techniques described herein may be combined with yearly or semi-annual veterinary visits for routine physical exams and would not require any additional invasive procedures (usually, blood samples are obtained for other tests). A positive test in the scenario of monitoring metastasis would prompt consideration of alternative therapies before the disease is so far advanced that no therapies are likely to provide benefit. These could include local irradiation at the site of metastasis, different chemotherapy protocols than those used in the initial treatment, targeted drugs, such as Palladia, immunotherapy, or investigational drugs that are safe and have mechanisms that reduce or eliminate cancer risk by attacking cancer-initiating cells or disrupting the tumor niche.

Some tests based on the techniques described herein may include exosome-based biological status determination. Exosomes are secreted, membrane-bound vesicles measuring 30 to 200 nM in diameter. They originate from the fusion of multivesicular endosomes to the plasma membrane. Like other microvesicles, exosomes carry cargo comprised of RNA, DNA, proteins, lipids, and cellular metabolites, but the loading of cargo into exosomes is an active process that does not reflect the cytoplasmic contents of the cell. Exosomes play pleiotropic roles in both physiological and pathological states of health. For example, exosomes have been reported to provide a cellular version of “wireless telegraph,” transmitting information locally, regionally, and distantly among disconnected cells within and between tissues. On the other hand, exosomes also appear to serve the function of cellular “dump trucks,” providing cells a mechanism to dispose of waste materials into the extracellular environment.

Exosomes can be powerful diagnostic tools, even if they are imperfect windows into cells. For example, the utility of serum exosomes as a diagnostic platform is dependent on their stability in biological fluids, their potential to be efficiently isolated, and the consistent and reliable presence of specific components in their cargo that are tightly associated with a disease state. On the other hand, the utility of serum exosomes as a diagnostic platform is independent of the source and function of such cargo. Enrichment of exosomes and/or comparably sized microvesicles from blood, plasma, and serum using instrumentation and methodology that is routinely available in diagnostic laboratories may enable applications of exosome diagnostics as a realistic goal. However, the identification of cargo originating from diseased cells (signal) from the background of normal exosomes (noise) is an issue associated with wide use of exosomes in clinical laboratory medicine. Even in the case of cancer where tumor cells release more exosomes than normal cells, the number of exosomes produced by 1×10⁹ cancer cells in a 1 cm³ tumor would be dwarfed by the exosomes produced by the patient's 4×10¹³ (40,000 times as many) normal cells. Stated differently, even if tumor cells produced on average 50,000-fold more exosomes than normal cells, about 50% of exosomes in serum would still be derived from normal cells, masking all but the strongest tumor-derived exosome (TEX) signals.

In contrast, the techniques described herein may enable virtually complete separation of TEX cargo and normal cell-derived exosome cargo using xenograft models and a novel bioinformatics pipeline. Such techniques may significantly reduce the number of patient samples needed to identify critical biomarkers of disease.

Exosome and machine-learning based techniques for analyzing and applying exosomal gene signatures to biological status determinations may be used in prognostic decision trees. In some examples, such techniques may help direct the type, dose, and intensity of therapy that is tailored to the molecular characteristics of a disease of a patient.

Techniques for exosome-based gene signature identification and the application of such gene signatures for machine-learning based techniques for biological status determinations, summarized here and detailed in the examples below, may include three steps that illustrate that xenograft models of osteosarcoma may enable discovery of exosome-based biomarkers that predict tumor biological behavior, arid thus may inform diagnosis arid/or determination of prognosis of osteosarcoma patients and patients that may be at risk for osteosarcoma.

In some examples, xenografts from multiple cell lines first may be established to obtain representative exosomes from tumors with different, albeit predictable biological behavior. The size of the experimental groups and the cross-species comparative approach described below with respect to a mouse host/dog donor osteosarcoma xenograft model may provide an accurate representation of the heterogeneity that exists in the disease. Genetically engineered tumor cell lines, reporter mice, and in vivo imaging may be used to define the creation of the metastatic niche and the establishment of pulmonary metastasis in individual mice. RNA may be isolated from serum exosomes collected longitudinally during the experiment and subjected to NGS.

The second step of some example techniques for exosome-based gene signature identification and the application of such gene signatures for machine-learning based techniques for biological status determinations summarized here and detailed in the examples below may include identifying conserved gene clusters that are associated with metastatic propensity. Such techniques use a hybrid genome comprised of donor and host genome built to identify species-specific, exosome associated transcripts originating from the tumor and from the host. Such data then may be used to identify co-regulated gene clusters (defined statistically by correlation analysis) that are conserved across species and are significantly associated with low or high metastatic propensity, and suitable candidates may be validated using qRT-PCR.

The third step is in species validation of exosome mRNA signatures in well-annotated samples (e.g., from children and dogs with osteosarcoma). Expression of aeries in candidate clusters may be quantified using qRT-PCR and/or NanoString quantitative nuclease protection assays, and the relationships between gene clusters and patient outcomes may be analyzed using unsupervised and/or supervised methods. Samples then may be divided into training and validation sets for machine learning algorithms to assign a probability for patterns to predict more aggressive (higher metastatic propensity), or less aggressive (lower metastatic propensity) osteosarcoma. The common rate of success in the validation set may be used to define the operating true positive (sensitivity) and true negative (specificity) of the test via receiver operating characteristic (ROC) curves. In such a manner, machine learning models may help enable biological status determinations for organisms of different types (e.g., human or non-human animal), which may help enable accurate, efficient, and/or early diagnosis or prognosis of a disease state or other physiological condition in the organism.

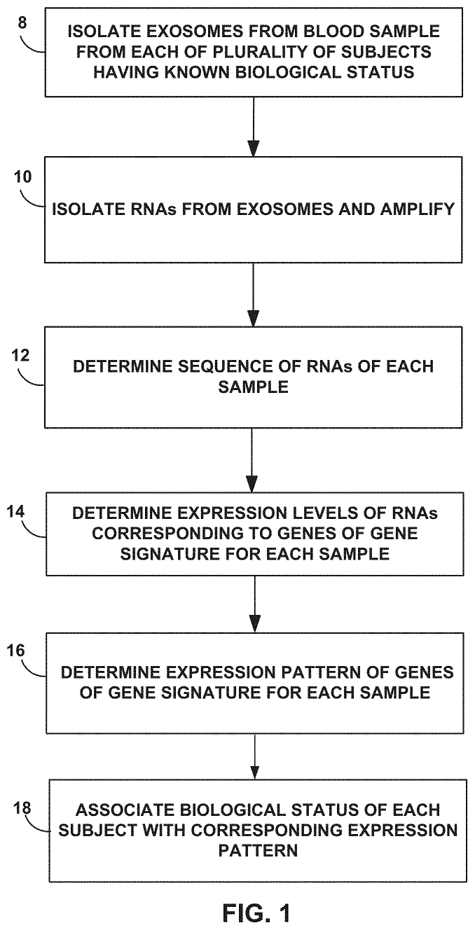

FIG. 1 is a flow diagram illustrating an example technique for training a machine learning model in accordance with the examples of this disclosure. According to the example of FIG. 1, a blood sample from each organism of a plurality of organisms is obtained, such as by using any suitable ones of the laboratory techniques described herein or any other suitable laboratory techniques. In some examples, one or more first organisms of the plurality of organisms have a known biological status that is different from a biological status (e.g., a known biological status) of one or more second organisms of the plurality of organisms. For example, the biological status of one or more first organisms of the plurality of organisms may be healthy, may have an existing disease state, may have a likelihood of developing the disease or a likelihood of progression of the disease state, or may have a different existing disease state, while the one or more second organisms may have at least one biological status (e.g., known biological status) that differs from at least one biological status of the one or more first organisms.

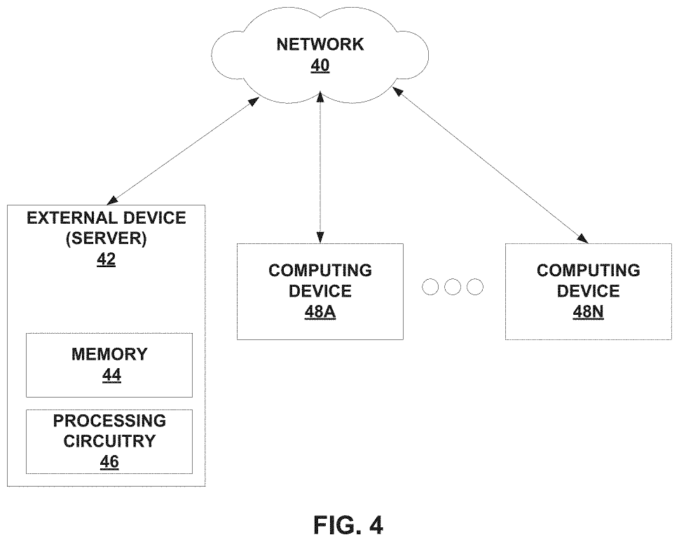

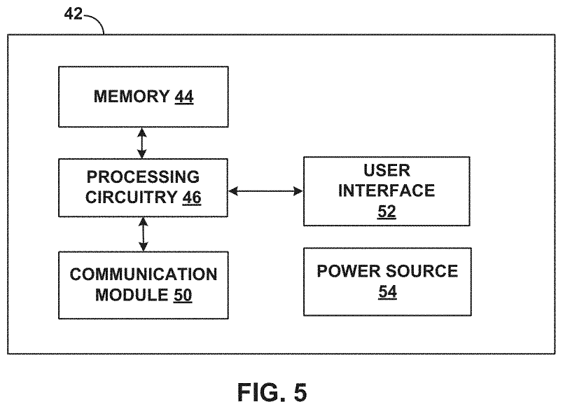

A plurality of exosomes from each of a plurality of samples of bodily fluid derived from corresponding ones of the plurality of organisms is isolated and amplified, such as by using any suitable ones of the laboratory techniques described herein (e.g., a PCR technique) or any other suitable laboratory techniques. In some examples, the plurality of exosomes from each of the plurality of samples of bodily fluid comprises a plurality of molecules RNA. For each of the plurality of samples of bodily fluid and for substantially each molecule of the plurality of molecules of RNA, a corresponding RNA sequence is determined. In some examples, the RNA sequences may be determined by processing circuitry of a computing device, such as one or more of the computing devices described below with respect to FIGS. 4 and 5. In some such examples, the computing device may be part of any suitable nucleotide-sequencing system.

For each of the plurality of samples of bodily fluid, the processing circuitry of the computing device determines, for each corresponding RNA sequence, whether the RNA sequence is associated with exactly one corresponding gene sequence of a gene signature comprising a plurality of gene sequences and determines, for each sample of the plurality of samples, an approximate number of times that each RNA sequence associated with exactly one corresponding gene of the gene signature occurs in the sample of bodily fluid. Determining the approximate number of times that each such RNA sequence occurs in the sample may provide an indication of an expression level exhibited by the organism of RNAs corresponding to the gene of the gene signature.

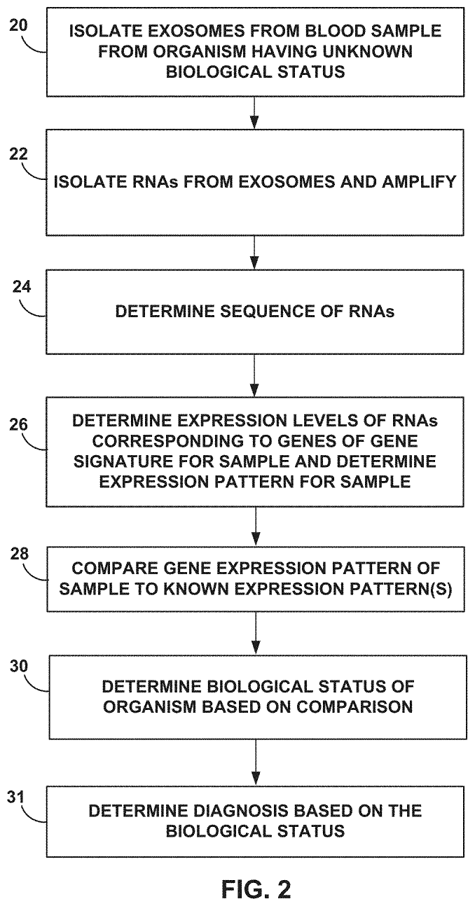

Processing circuitry (e.g., the processing circuitry described above or other processing circuitry) then determines, for each sample of bodily fluid and using one or more machine learning models, a pattern of expression of the plurality of gene sequences of the gene signature associated with the sample of bodily fluid based on the approximate number of times that each RNA sequence associated with exactly one corresponding gene of the gene signature occurs in the sample of bodily fluid. Next, using the one or more machine learning models and for each organism (e.g., subject) of the plurality of organisms, the processing circuitry associates the biological status of the organism with the corresponding pattern of expression of the of the plurality of gene sequences of the gene signature associated with the sample of bodily fluid from the organism. In this manner, the technique of FIG. 1 may train the one or more machine learning models to determine an unknown biological status of a sample obtained from a test organism, as described below with respect to FIG. 2.

FIG. 2 is a flow diagram illustrating an example technique for determining a biological status (e.g., a previously unknown biological status) of an organism in accordance with the examples of this disclosure. In some examples, determining an unknown biological state of an organism may include determining a likelihood that the organism has or may develop a particular disease (e.g., osteosarcoma or other cancer) or other physiological condition, a progression or likelihood of progression of an existing disease, other behavior of an existing disease, or other statuses that may be associated with the disease or condition. Expression levels of genes (either absolute or relative to other genes) associated with a gene signature that corresponds to the disease or other condition may enable techniques for blood-based testing to determine such biological states. In order to determine expression levels of genes of a gene signature from a blood sample (or sample of other bodily fluid), RNA molecules may be isolated from exosomes isolated from the sample. Machine learning models may be used to analyze abundances of the RNA molecules having sequences associated with sequences of each of the genes of the gene signature (e.g., by qRT-PCR) may enable determination of expression levels of the genes for a particular organism, as discussed below with respect to the example technique of FIG. 2.

In one example, the gene signature (e.g., an exosomal gene signature) is a plurality of genes associated with osteosarcoma: SKA2, NEU1, PAF1, PSMG2, and NOB1. These five genes may be a selected subset of a larger plurality of genes associated with osteosarcoma, such that other genes from the larger plurality of genes may be used to diagnose osteosarcoma in other examples. Determination of expression levels of each of these five genes (e.g., absolute expression levels or expression levels relative to other genes of the gene signature) may enable determination of whether an organism (e.g., a dog, other non-human animal, or human) is at risk for developing osteosarcoma, has osteosarcoma, and/or, in the case of existing osteosarcoma, a likelihood that the disease may progress relatively more or less aggressively. Determining a biological status corresponding to osteosarcoma in an organism by analysis of SKA2, NEU1, PAF1, PSMG2, and NOB1 expression levels may help enable earlier and/or more accurate diagnosis or determinations of prognosis, and in sonic examples may help inform decisions regarding treatments.

According to the example of FIG. 2, a plurality of exosomes from a sample of bodily fluid derived an organism is obtained, such as by using any suitable ones of the laboratory techniques described herein or any other suitable laboratory techniques. In some examples, the plurality of exosomes comprises a plurality of molecules of RNA. Next, the plurality of molecules of RNA are isolated from the exosomes and amplified using any suitable technique, such as a PCR technique.

For each substantially each molecule of the plurality of molecules of RNA, a corresponding RNA sequence is determined. In some examples, the RNA sequences may be determined by processing circuitry of a computing device, such as one or more of the computing devices described below with respect to FIGS. 4 and 5. In some such examples, the computing device may be part of any suitable nucleotide-sequencing system. Next, the processing circuitry of the computing device determines, for each corresponding RNA sequence, whether the RNA sequence is associated with exactly one corresponding gene sequence of a gene signature comprising a plurality of gene sequences and determines an approximate number of times that each RNA sequence associated with exactly one corresponding gene of the gene signature occurs in the sample of bodily fluid. Determining the approximate number of times that each such RNA sequence occurs in the sample may provide an indication of an expression level exhibited by the organism of RNAs corresponding to the gene of the gene signature.

Processing circuitry (e.g., the processing circuitry described above or other processing circuitry) then analyzes, using one or more machine learning models, the approximate number of times each RNA sequence associated with the exactly one corresponding gene of the gene signature occurs in the sample of bodily fluid and determines a pattern of expression exhibited by the organism of the plurality of gene sequences of the gene signature associated with the sample of bodily fluid based on the approximate number of times that each RNA sequence associated with exactly one corresponding gene of the gene signature occurs in the sample of bodily fluid.

Next, using the one or more machine learning models, the processing circuitry compares the pattern of gene expression of the organism to at least one known pattern of gene expression (e.g., one of the known patterns of gene expression described above with respect to the technique of FIG. 1) and determines the biological status of the organism based on the comparison. The processing circuitry may then determine a diagnosis for the organism that is based on the biological status. The diagnosis may be an identification of a disease or non-diseased state (e.g., positive for osteosarcoma or negative for osteosarcoma) or a risk of contracting a disease or condition (e.g., a high risk of developing a condition or a low risk of developing a condition). In this manner, the technique of FIG. 2 may use one or more trained machine learning models to determine an unknown biological status of a sample obtained from a test organism, and, in some examples, determine a diagnosis or risk of contracting the condition according to the biological status.

As discussed above, osteosarcoma is an incurable, highly metastatic bone tumor that primarily affects young children and adolescents; interestingly, it is among the most common tumors affecting dogs. The 5-year survival rate for human patients with localized disease is 60-70%; however, more than half of patients relapse and die from metastatic disease within 10-years of diagnosis. Aggressive treatments to prevent or delay metastasis of osteosarcoma are critical for long-term survival; however, the intrinsic properties of the tumor are also major determinants of outcome, creating opportunities for personalized therapies. The need for personalized medicine is clearly exemplified by the heterogenous nature of osteosarcoma as well as the concern over therapy related toxicity in pediatric oncology. However, we still struggle to provide accurate, reproducible prognostic tests that can be readily translated to guide therapy in the clinical setting.

Non-invasive tests that inform prognosis and longitudinal remission status are persistent unmet needs for osteosarcoma, and in order for these tests to be successful, it may be helpful to uncover specific prognostic biomarkers that can inform risk, early detection, response to therapy, and progression. Circulating cell-free nucleic acids have been explored as potential sources of biomarkers in osteosarcoma, as well as microRNA (miRNA) and non-coding RNAs. Additionally, the discovery of exosomes and their role in transferring genetic information between cells has sparked interest in utilizing these extracellular vesicles in the discovery of key genes promoting tumor progression.

Exosomes are extracellular vesicles, approximately 30-200 nm in size that are released by virtually every cell type. Diseased organs and abnormal cells, such as tumor cells, will generate a prolific number of exosomes which carry biological information such as nucleic acids and proteins, through the circulation to distant organ sites. Moreover, exosomes are thought to play an important role in promoting a more favorable tumor microenvironment, which may be essential for the dissemination and metastasis of certain tumors, including osteosarcoma. Exosomes are easily accessible from bodily fluids, such as blood and urine, making them desirable biomarker candidates that have been explored in many cancer types but have not been investigated in osteosarcoma.

A system can recapitulate the heterogeneous biological behavior of osteosarcoma in a mouse xenograft model. Using novel bioinformatics methods to study tumor-stromal interactions in these models, these two molecular subgroups promote formation of different tumor associated stromal environments. As described herein, exosomes isolated from osteosarcoma cell lines will alter gene expression in target cells, both in vitro and in vivo in xenograft mouse models. Bioinformatics pipelines enable virtually complete separation of tumor-derived exosome cargo from host-cell derived exosome cargo. The tumor-derived exosomes contain unique mRNA profiles that could be used to identify OS, and a unique gene signature is trainable in machine learning models to establish the presence of osteosarcoma in dogs using blood samples, for example.

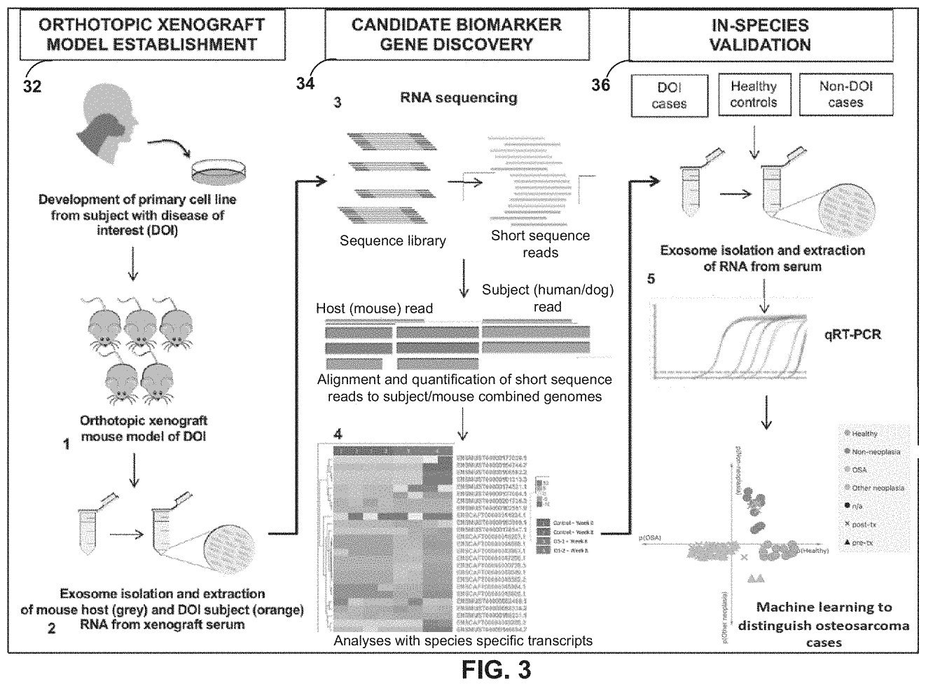

FIG. 3 is flow diagram illustrating an example technique for orthotopic xenograft model establishment, candidate biomarker gene discovery, and in-species validation of a gene signature that includes biomarker genes in accordance with the examples of this disclosure. The technique illustrated in FIG. 3 may provide an enabling platform for biomarker discovery using species-mismatched xenografts, where the biomarker may be indicative of a disease of interest (DOI) such as osteosarcoma or other types of cancer, in particular, this model may enable biomarker discovery of biomarkers derived from mRNA loaded in exosomes from these samples. As illustrated in FIG. 3, orthotopic xenografts may be created to generate species-mismatched exosomes in a single environment. Serum exosomes may be collected using established methods without need for subset enrichment, such as by affinity labeling, which may add variability to the process. Controlled and reproducible methods for low-input library creation and NGS may be used to capture the complete profile of exosome mRNAs without a need to separate donor-derived (e.g., tumor) exosomes from host-derived exosomes.

The example bioinformatics pipeline illustrated in FIG. 3 may be used to identify donor- and host-derived exosome mRNAs. In some examples, co-regulated gene clusters may be aggregated to define biomarkers, which increases available power by limiting multiple testing. In some examples, the use of gene clusters may reduce the complexity of heterogeneous diseases, a goal that is unachievable with single biomarkers, and/or may reduce the reliance on any single mRNA. The platform for biomarker discovery illustrated in FIG. 3 may be done relatively rapidly and/or inexpensively in some laboratory models, identifying cancer biomarkers that include both TEX mRNAs and host-derived non-TEX mRNAs associated with a disease, the intrinsic host response, or response to specific therapy.

The example method of FIG. 3 may further include in vivo validation of biomarkers that are reliable and reproducibly produced and secreted by donor cells (i.e., the tumor) or as part of the host response. In vivo validation may be done prospectively or retrospectively. As described herein, machine learning allows for use of relatively smaller sample sizes than would be needed using de novo discovery methods, such as identification of biomarkers from clinical trials samples, which may increase speed and/or reduce cost. For example, applying the biomarkers of each sample to one or more machine learning models to distinguish subjects likely having the DOI (e.g., osteosarcoma) from health subjects and/or conditions other than the DOI. The preliminary data described herein and illustrated in the following figures provides an example of how biomarkers that are associated with the presence of osteosarcoma in dogs may be identified as proof of concept for the platform.

More specifically, the following example method may be used to obtain exosomes, sequence obtained mRNA from the exosomes, and determine levels of expression of genes associated with the obtained mRNA. In addition, machine learning models may be trained to classify the quantified levels of mRNA from exosomes in the obtained samples.

For cell culture, two canine osteosarcoma cell lines, representing previously described “highly aggressive” and “less aggressive” molecular phenotypes (OS-1 and OS-2), were used in the study described below. OS-1 and OS-2 are derivatives of the OSCA-32 and OSCA-40 cell lines, respectively. OS-1 and 0S-2 cells were modified to stably express green fluorescent protein (GFP)a and firefly luciferase and used for orthotopic injections in mice. Prior to mouse injections, cells were grown in exosome-depleted DMEM media (DMEM with 5% glucose and L-glutamine, supplemented with 10% exosome-depleted FBS Media Supplement—USA Certified, 10 mM 4-(2-hydroxyethyl)-1-piperazine ethanesulphonic acid buffer (HEPES) and 0.1% Primocin), and cultured at 37° C. in a humidified atmosphere of 5% CO2. Each cell line was passaged more than 15 times before the experiments; however, cell lines were repeatedly authenticated to ensure short tandem repeats were conserved to the original tumor material from which they were derived, as well as to established signatures from the original established cell lines. The parental canine osteosarcoma cell lines (OSCA-32 and OSCA-40) are available for distribution through Kerafast, Inc.

With regard to tumor xenografts, six week-old, female, athymic nude mice (strain NCr nu/nu) were obtained from an approved vendor. Animals were assigned to separate cages in random order for each experiment. All mouse experiments were approved by The University of Minnesota Institutional Animal Care and Use Committee (Protocol No.: 1307-30806A). Mice were anesthetized with xylazine (10 mg/kg, intraperitoneally (I.P.)) and ketamine (100 mg/kg, I.P.) in preparation for intratibial (IT) injections. Canine osteosarcoma cells were suspended in sterile PBS, and 10 μl containing 1×10⁵ cells was injected I.T. Control mice had 10 μl sterile PBS injected I.T. All injections were administered into the left tibia using a tuberculin syringe with 29-gauge needle. For each osteosarcoma cell line, OS-1 and OS-2, 5 mice received cell-I.T. injections and 3 mice received PBS-I.T. injections. Buprenorphine (0.075 mg/kg, I.P. every 8 hours) was administered for analgesia for 24 hours following the injections, and prophylactic ibuprofen was administrated in the water for the next 3 days.

Mice were monitored by weekly bioluminescence imaging and tumor size measurements. Blood was collected into BD microtainer serum separator tubes by facial vein phlebotomy from all mice at 2, 4, 6, and 8 weeks after the injections. Microtainer tubes were centrifuged at 3,000×g for 15 minutes and approximately 250 μl pooled serum was collected from all the mice in each cage. Serum was stored at −80° C. until analysis. At 8 weeks after the injections, the mice were humanely euthanized using a barbiturate overdose. Blood was collected via intracardiac phlebotomy. The tibiae and the lungs were collected from mice injected with osteosarcoma cells (n =10) and placed in 10% neutral buffered formalin for histopathology or stored at −80° C. There were no grossly visible tumors noted in the pulmonary tissue.

Next, exosomes were precipitated from serum samples from control mice and from tumor bearing mice at week 8 using ExoQuick reagent according to the manufacturer's instructions. Briefly, serum was mixed with ExoQuick reagent at a volume of 252 μl ExoQuick per 1 ml of serum. The mixture was incubated for 30 minutes at 4° C., followed by centrifugation at 1,500×g for 30 minutes to precipitate exosomes. The resulting supernatant was removed and discarded, and the tubes were centrifuged for an additional 5 minutes at 1,500×g to remove any remaining supernatant. Exosomal RNA was extracted using SeraMir ExoRNA. Amp Kit, according to the manufacturer's instructions,

Two technical replicates from each sample were sequenced and analyzed independently. Sequencing libraries were prepared using the Clontech SMARTert Stranded Total RNA-Seq Kit v2—Pico Input Mammalian kit. RNA sequencing (50-bp paired-end, with HiSeq 2500 Illumina) was performed at the University of Minnesota Genomics Center (UMGC). A minimum of sixteen million read-pairs was generated for each sample and the average quality scores were above Q30 for all pass-filter reads.

Initial quality control analysis of RNA sequencing FASTQ data was performed using FastQC software. FASTQ data were trimmed with Trimmomatic. Kallistop was used for pseduoalignment and quantifying transcript abundance. For accurate alignment of sequencing reads to canine and murine genes within xenograft tumors, a kallisto index was built from a multi-sequence FASTA file containing both the canine (CanFam3.1) and murine (GRCm38.p5) genomes. For each species, transcripts <200 bp were removed from the FASTA files. The masked FASTA files were then merged for a total of 121,749 murine and canine transcripts. Insertion size metrics were calculated for each sample using Picard software. Data will be deposited in GenBank/GEO.

The ‘DESeq2’ package in RStudio was used for differential analysis of transcript counts obtained from kallisto data. Transcript counts were first summarized to gene counts and then DESeq2 was used to convert count values to integer mode, correct for library size, and estimate dispersions and log2 fold changes between comparison groups. Genes with a Benjamini-Hochberg adjusted p-value<0.05 and log2 fold change>+/−4 between control and xenograft samples were considered significantly differentially expressed genes (DEGs). Statistically differentially expressed canine genes were removed if they had a DESeq2 normalized value of greater than zero in the control (mouse sequences) as these would be genes that are highly homologous between the mouse and dog.

Counts per million (CPM) values of genes were log2 transformed and mean centered prior to clustering. The ComplexHeatmap package was used for clustering and creating heatmap figures. Enriched pathway and functional classification analyses of DEGs were performed using QIAGEN's Ingenuity® Pathway Analysis (IPA®). The reference set for all IPA analyses was the ingenuity Knowledge Base (genes only) and canine associated gene names were used as the output format for input datasets with canine genes and murine associated gene names were used as the output format for input datasets with murine genes.

Next, qRT-PCR was validated for sequencing data. Serum or plasma samples were obtained from client-owned dogs with naturally-occurring osteosarcoma before and after treatment as part of routine biobanking efforts. The samples included in the analysis were identified retrospectively. Serum samples were also obtained from client-owned dogs that were hospitalized with various non-malignant conditions. Serum samples were obtained from healthy staff- and student-owned dogs. Blood was collected into vacutainer tubes that were centrifuged at 3,000×g for 15 minutes. Aliquots of serum or plasma were transferred to 1.5 ml microcentrifuge tubes and stored at −80° C. until analysis. All treatment decisions were at the discretion of the attending clinician.

Exosomes were precipitated from canine serum or plasma samples using ExoQuick reagent according to the manufacturer's instructions. Additional steps were included for plasma samples: 10 μl of thrombin was added for each 1 ml of plasma. The sample was then mixed at room temperature for 5 minutes, followed by centrifugation at 10,000 rpm for 5 minutes. The supernatant was transferred to a new microcentrifuge tube, and the volume recovered was noted. Plasma and serum samples were then treated the same. Briefly, the sample was mixed with ExoQuick reagent at a volume of 252 μl ExoQuick per 1 ml of serum. The mixture was incubated for 30 minutes at 4° C., followed by centrifugation at 1,500×g for 30 minutes to precipitate exosomes. The resulting supernatant was removed and discarded, and the tubes were centrifuged for an additional 5 minutes at 1,500×g to remove any remaining supernatant. Exosomal RNA was extracted using the mirVana miRNA Isolation Kit, according to the manufacturer's instructions.

Elimination of genomic DNA and reverse transcription were both carried out using QuantiTect Reverse Transcription Kit. Real-time quantitative reverse transcriptase PCR (qRT-PCR) was performed on a LIGHTCYCLER 96u with FastStart SYBR Universal Green Master Mixv Protocol. GAPDH was used as the reference standard for normalization and relative levels of steady state mRNA were established using the comparative [delta]Ct method. The relationship between RNA-sequencing data and qRT-PCR values for the transcripts of interest were analyzed using Pearson's correlation.

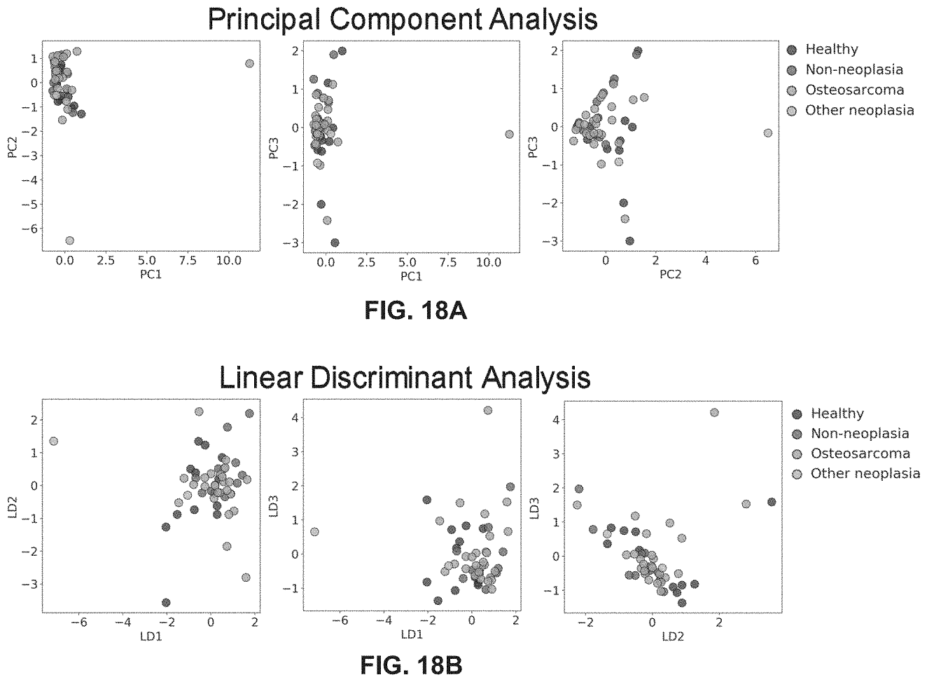

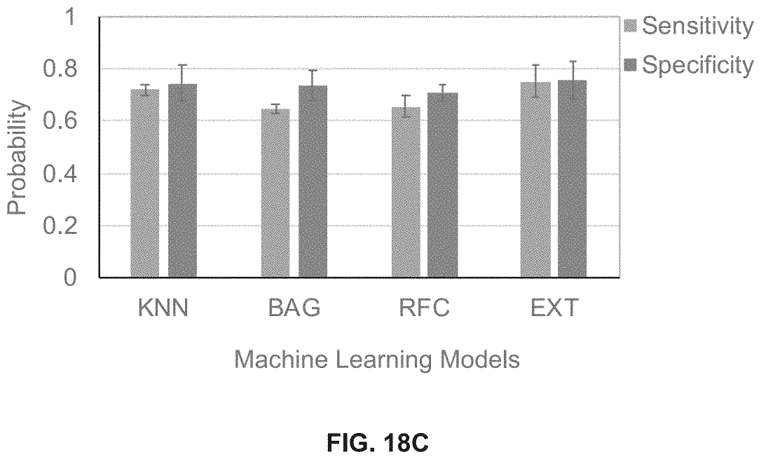

Machine learning was then performed using the levels of expression from the samples, e.g., qRT-PCR values for the obtained transcripts from the exosomes. Gene expression data from healthy (n=13), non-neoplasia (conditions other than cancer; n=10) osteosarcoma (OS; n=27), and other neoplasia (non-OS cancers; n=2) pre-treatment samples (52 total) were standardized by resealing to a mean of zero and a standard deviation of one for each of the five genes. The results of the following methods are shown in more detail in FIGS. 18A-23B. Principal component analysis (PCA) and linear discriminant analysis (LDA) were used initially to assess how well the samples were separated across the four categories. Different machine learning algorithms were then used to build different machine learning models, including Logistic Regression (LR), Linear Discriminant Analysis (LDA), k-Nearest Neighbors (KNN), Decision Tree Classifier (CART), Gaussian Nave Bayes (NB), Support Vector Machine (SVM), Bagging (BAG), Random Forest (RF), Extra Trees Classifier (EXT). Adaptive Boosting (ADA), Stochastic Gradient Boosting (SGB), and. Neural Network (NN) algorithms. For training and optimization, data were randomly split into training and validation sets using K-fold cross-validations with sample stratification (when possible). K-fold cross-validation randomly splits data into K groups, where K-1 groups were used for training and one remaining group was used for validation; repeated for K times with each of K validation sets being used only once. K-fold cross-validations were then repeated ten times to ensure performance stability across multiple tests. Classification scores were then averaged over 10 repeated cross-validations. Classification scores of learning models were compared individually and in combination with LDA transformation. In the case of LDA transformation, expression data were first fit and transformed using three-component LDA prior to machine learning cross-validation tests. Top models with the best averaged sensitivity and specificity were chosen for further optimization and model deployment.

Four top-performing learning models (e.g., KNN, BAG, RF, and EXT) with three-component LDA transformation were chosen for deployment and predictive classification. Data from the four categories (with known disease states) were fit and transformed with three-component LDA for training of the four learning models. Unknown samples (post-treatment OS subjects) were transformed based on the fitted training set and classified using the four trained learning models. Results from the prediction calls were further tested against survival data of the post-treatment OS subjects over time as a means for detecting residual disease, as shown in FIGS. 23A and 23B.

FIG. 4 is functional block diagram illustrating an example configuration of a computing system that may be used to implement the machine learning model training and biological status determination described herein. FIG. 5 is a functional block diagram illustrating an example configuration of a computing device of the computing system of FIG. 4 that may be used to implement the machine learning model training and biological status determination described herein.

As illustrated in FIGS. 4 and 5, various aspects of the techniques described herein may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In the example illustrated in FIG. 4, one or more computing devices 48A-48N are connected to a network 40. In some examples, an external server device, such as server device 42, may also be connected to network 40. The server device 42 shown in FIGS. 4 and 5 may include processing, circuitry 46, memory 44, user interface 52, communication module 50, and power source 54. Processing circuitry 46 may include one or more processors. In one example, processing circuitry 46 is configured to run the software instructions in order to control operation of system 218. Processing circuitry 46 can include one or more processors, including one or more microprocessors, digital signal processors (DSPs), application specific integrated circuits (ASICs), field programmable gate arrays (FPGAs), or arty other equivalent integrated or discrete logic circuitry, as well as any suitable combination of such components. The term “processor” or “processing circuitry” may generally refer to any of the foregoing logic circuitry, alone or in combination with other logic circuitry, or any other equivalent circuitry.

Memory 44 may include any volatile or non-volatile media, such as a random access memory (RAM), read only memory (ROM), non-volatile RAM (NVRAM), electrically erasable programmable ROM (EEPROM), flash memory, and the like. As mentioned above, memory 44 may store information including instructions for execution by processing circuitry 46 such as, but not limited to, instructions for performing the techniques described herein. Communication module 50 may provide one or more channels for receiving and/or transmitting information. Communication module 50 may be configured to perform wired and/or wireless communication with other devices, such as radio frequency communications. In other examples, communication module 50 may not be implemented, and instead, memory 44 may be removable (e.g., a removable flash memory).

Power source 54 delivers operating power to various components of computing device 218. Power source 54 may generate operational power from an alternating current source (e.g., residential or commercial electrical power outlet) or direct current source such as a rechargeable or non-rechargeable battery and a power generation circuit to produce the operating power. In other examples, non-rechargeable storage devices may be used for a limited period of time.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an MID, an external programmer, a combination of an Imp and external programmer, an integrated circuit (IC) or a set of ICs, and/or discrete electrical circuitry, residing in an IMD and/or external programmer.

Further aspects of the disclosure will now be discussed, including further details of the techniques described herein. It is contemplated that the example laboratory techniques described for accomplishing routine laboratory tasks, such as the collection of blood and the isolation of serum from blood, as well as others, are not intended to be limiting and may be performed by any suitable laboratory techniques. In addition to the techniques described above, supplementary techniques, as described below, may be employed. Organisms other than mice, dogs, and/or humans may be used in some applications of the example techniques herein, as is true for tumors other than osteosarcoma, or even tissues used in organ transplantation such as heart, liver, kidney, or lungs, whereby markers can be identified to determine likelihood of transplant acceptance or rejection. Additionally, or alternatively, different gene signatures having any number of any particular genes and being associated with any particular disease state or other physiological condition in some applications of the techniques described herein.

The following example techniques involve creating osteosarcoma models with distinct metastatic propensity for exosome biomarker discovery. Tumor cells secrete large numbers of exosomes, and the content of these TEX can provide insights into the tumor's growth rate and metastatic propensity. But finding the specific markers that allow us to distinguish among tumor (or patient) groups is challenging, both because of inter-patient heterogeneity and because of the background noise from exosomes in the blood that do not come from the tumor. The principle to use xenograft models to identify donor and host species, combined with the choice of using mRNA biomarkers, represents an improvement in the benefits of xenograft techniques. Specifically, mRNAs meeting the criteria for species-specific separation by sequencing (an estimated more than 80% of transcripts may be assigned to one species through the methodology described herein), and because many genes are co-regulated transcriptionally or post-transcriptionally, clustering methods may be used to increase power and reduce uncertainty introduced by multiple testing. That is, for example, 60 markers that move together may be more robust than any single one of those markers in isolation. These markers that move together may be coordinately regulated (i.e., co-regulated genes) that can provide greater assurance that the disease is present when compared to detecting a few or only one biomarkers that may be subject to periodic absence from the sample. Given the genomic instability of the tumors, there may be high tolerance for loss of genes in a cluster without losing power of classification for any sample. Other exosome-associated biomolecules pose issues. For example, there are no tumor-associated recurrent mutations in osteosarcoma, so building bait panels for cell-free DNA or to identify mutations in exosome DNA may be challenging. In some examples, the sequence similarity among microRNAs among species may make it challenging to separate donor from host species, at least in the case of humans, dogs, and mice, which are common osteosarcoma model species. In some examples, the mechanisms that regulate protein synthesis, stability, and distribution may mask co-regulated proteins, which may reduce the strength of cluster analysis in proteomes.

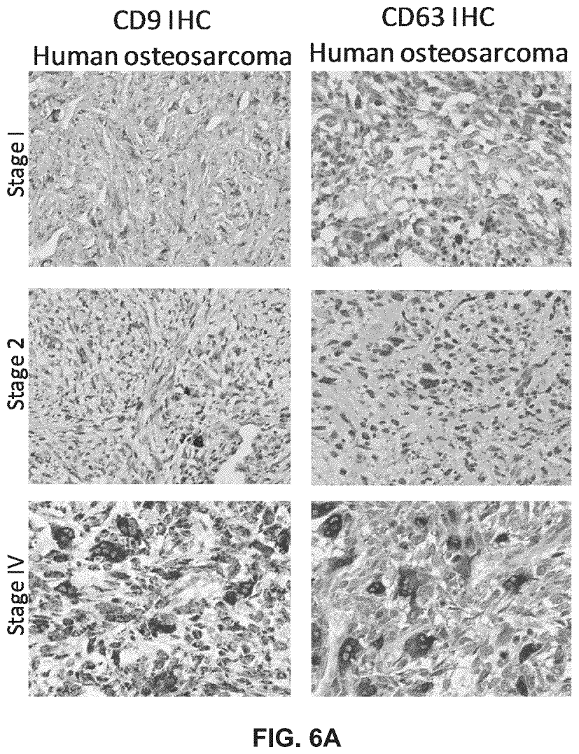

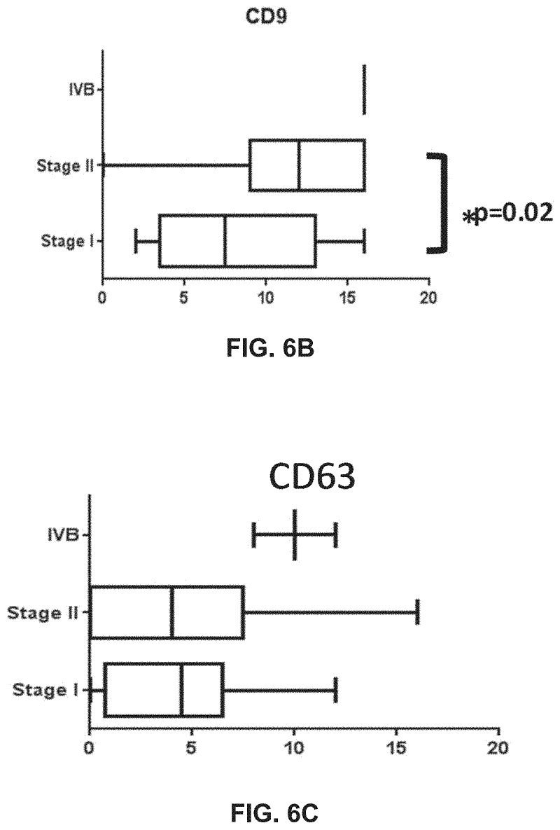

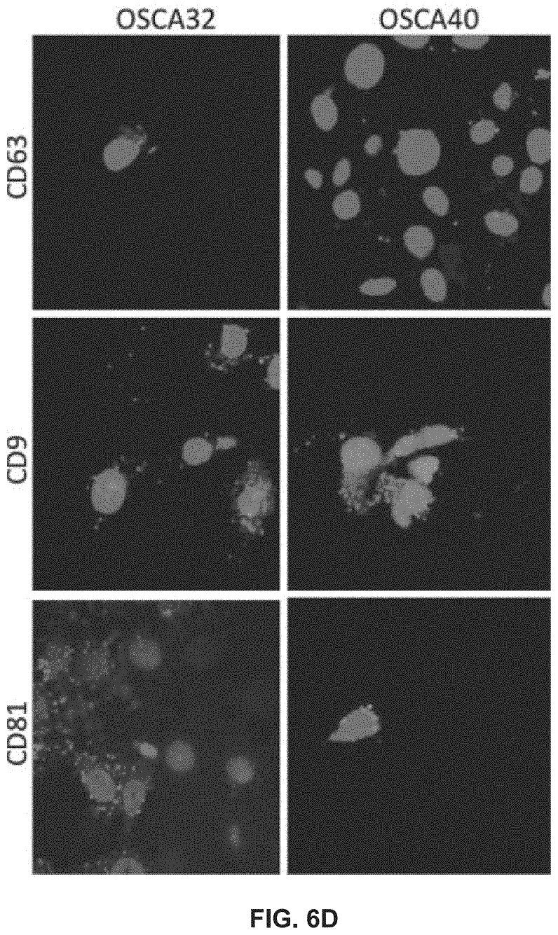

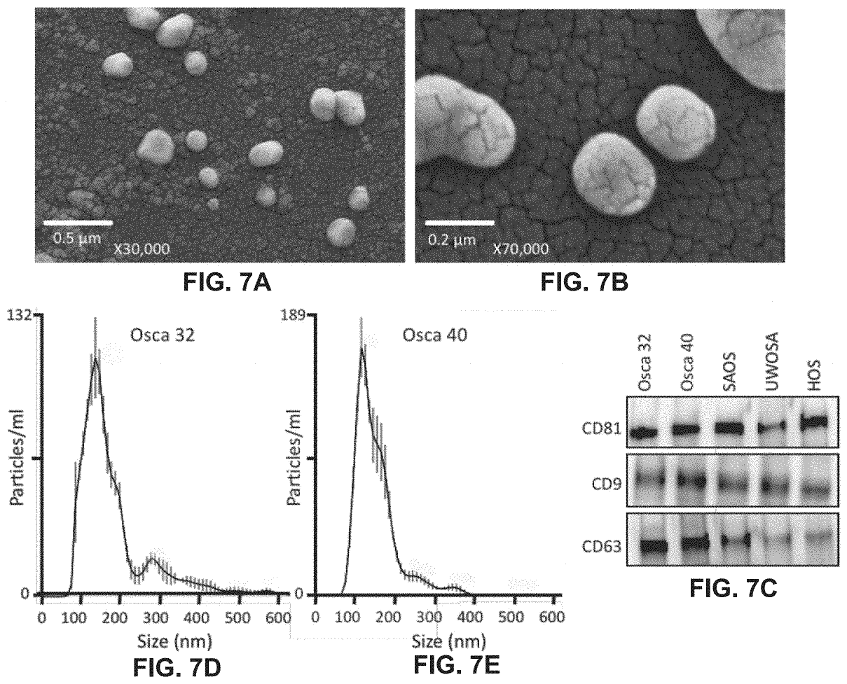

FIGS. 6A-7E illustrate example physical and biochemical characterizations of osteosarcoma exosomes. FIG. 6A includes digital images from exosome specific staining in human osteosarcoma tissues. Dark areas indicate positive detection of exosomes in the three images for each of CD9 and CD63 markers for respective Stage I, II, and IV of osteosarcoma.

FIGS. 6B and 6C are graphs of expression levels for different markers across different stages of osteosarcoma. Tissue biopsy samples from human osteosarcoma patients were stained for the presence of exosome markers CD9 and CD63. Box and whisker plots indicate the THC expression level of the two exosome markers across stage I, II, and IV osteosarcomas. FIG. 6D is digital images of exosomes isolated from different osteosarcoma cell lines in accordance with examples of this disclosure, and illustrates cultured dog osteosarcoma cell lines OSCA-32 and OSCA-40 that were stained for exosome-associated tetraspanins CD63, CD9, and CD81 by immunofluorescence. Exosomes provide a means for communication between cells at distant sites. Transcriptomic profiles of exosomes can be characterized by next generation RNA sequencing.

FIGS. 6A-9 show that microvesicles released from osteosarcoma cells display physical properties consistent with exosomes. Exosomes were visualized in human osteosarcoma biopsy samples as well as cultured dog osteosarcoma cell lines by immunostaining for exosome-specific tetraspanins CD9, CD63 and CD81 (FIGS. 6A-6D). Representative images from human osteosarcoma tissue samples taken at various stages of disease demonstrate increased staining of exosome specific markers CD9 and CD63 in higher grade tumors. Data from 80 human osteosarcoma biopsy samples showed statistically significant greater CD9 expression in stage-2 tumors (high grade, localized disease) compared to stage-1 tumors (low grade, localized disease). In addition to fixed tissue samples, we also detected exosome production from cultured canine osteosarcoma cell lines OSCA-32 and OSCA-40 using immunofluorescence staining for CD63, CD9 and CD81. Collectively, these results suggest that osteosarcoma cells are prolific exosome producers and the number of exosomes produced may increase with disease severity.

FIGS. 7A and 7B are SEM images, at different magnification levels, of representative exosomes isolated from OSCA-40 cell lines in accordance with examples of this disclosure. The physical properties of exosomes enriched from canine osteosarcoma cell lines were established, and from serum samples of healthy dogs and dogs with osteosarcoma using scanning electron microscopy (SEM), NanoSight particle tracking analysis and immunoblotting (FIGS. 7A-9). SEM showed the size and shape of the vesicles was consistent with that predicted for exosomes. Nanoparticle tracking showed the mean vesicle size ranged from of 149 nm-180 nm with a mode of 117 nm-132 nm, which is also consistent with the expected size of exosomes (FIGS. 7D-3 and FIGS. 8A-8F). Finally, the osteosarcoma cell line-derived exosomes showed enrichment of CD63 and depletion of β-actin. Serum exosomes were similar in size to cell line-derived exosomes, supporting the methodology used for isolation from both sample types.

Osteosarcoma cells are prolific exosome producers. One method of enriching exosomes includes the use of a modified version of System Biosciences' (SBI's) Exoquick, which uses a proprietary formula to aggregate membrane bound microvesicles in the size range of exosomes so they can be precipitated out of solution. To further improve the efficiency and reduce the cost of this method, a centrifugation and clearing step may be added for isolation of exosomes from cell culture, serum, or plasma. Additional steps may be undertaken for isolation from plasma to remove clotting factors that might affect the performance of Exoquick.

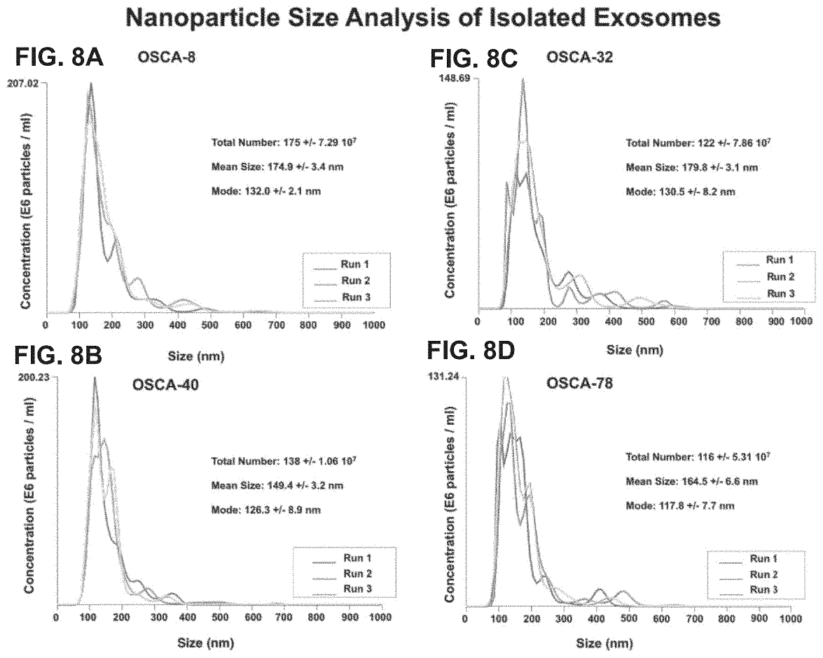

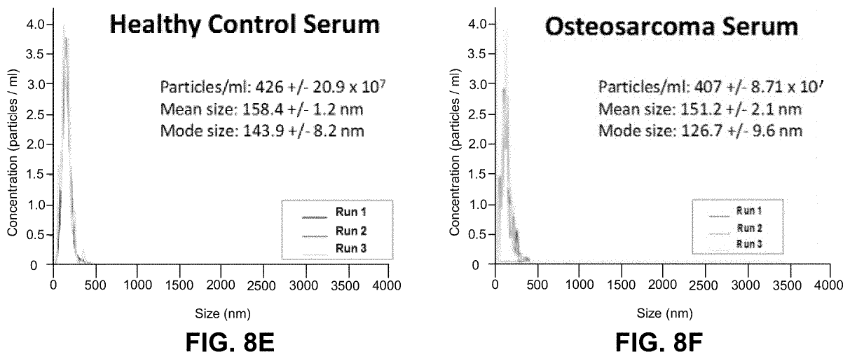

FIG. 7C is digital images of results of a Western blot assay illustrating quantities of three expression products isolated from each of five different cell lines in accordance with examples of this disclosure. FIG. 7C shows enrichment of tetraspanin proteins, CD9, CD63, and CD81, which are enriched in exosomes. Thus, detectable (e.g., abundant) expression may be taken as a biochemical definition of exosome enrichment. The size distribution of exosomes isolated from osteosarcoma cells using this isolation method, as well as their physical and biochemical characteristics, are shown in FIGS. 7D-8D. The results are representative of data reproducibly generated by more than twelve scientists across multiple labs.

FIGS. 7D and 7E are graphical representations of exosome size nanometers (nm)) and exosome quantification (in particles per milliliter (mL) of solution) of exosomes isolated from OS32 and OS40 cell lines in accordance with examples of this disclosure. FIGS. 8A-8D are graphical representations of exosome size (in nm) and exosome quantification (in particles per mL of solution) of exosomes respectively isolated from osteosarcoma cell lines OSCA-8, OSCA-40, OSCA-32, and OSCA-78 cell lines, in accordance with examples of this disclosure. In the example of FIGS. 8A-8D, the exosome, samples were analyzed using Nanoparticle Tracking Analysis. Samples of exosomes were resuspended in 700 microliters (μL) phosphate-buffered saline (PBS), diluted 1:10, and analyzed using a NanoSight LM10. Three runs for each of the OSCA-8, OSCA-40, OSCA-32, and OSCA-78 samples are depicted. Total particle number, mean particle size, and mode are shown for each sample, showing modal diameters of approximately 130 nm. Similar results were obtained in human and mouse osteosarcoma cell lines. In the example of FIGS. SE and 8F, NanoSight particle tracking analysis of triplicate samples serum from a healthy control and osteosarcoma (OSA) positive representative canine patients showing modal diameters of approximately 130 nm.

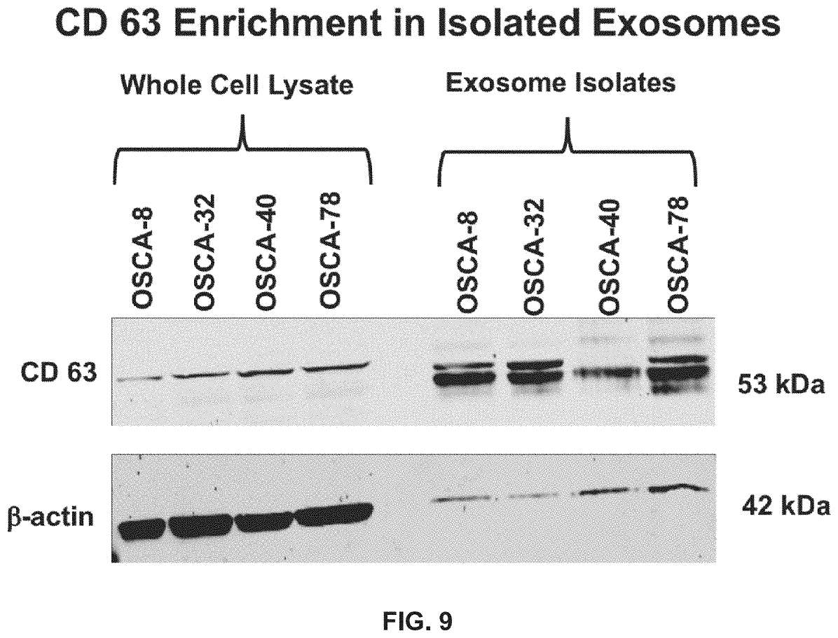

FIG. 9 is digital images of results of a Western blot assay illustrating immunoblotting of exosome preparations documenting enrichment of tetraspanins (CD63) and depletion of β-actin in OSCA-8, OSCA-32, OSCA-40, and OSCA-78 dog osteosarcoma cell lines. The comparison of the ratio of CD63/β-actin for each cell line between cellular and exosome extracts may be significant.

As illustrated in FIG. 9, depletion of β-actin, which here was used to show exosome enrichment, may be much less abundant in exosomes, so the ratio of CD63 to β-actin in cell lysates is very low, and the ratio of CD63 to β-actin in exosomes will be very high. That inverse relationship in illustrated in the Western blot of FIG. 9. The inverse relationship between CD63 and β-actin is confirmation that the enrichment process worked. CD63/β-actin ratio may be low in cellular extracts and high in exosome extracts.

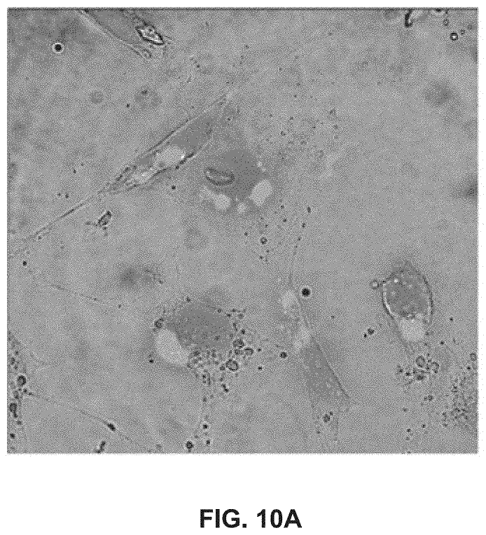

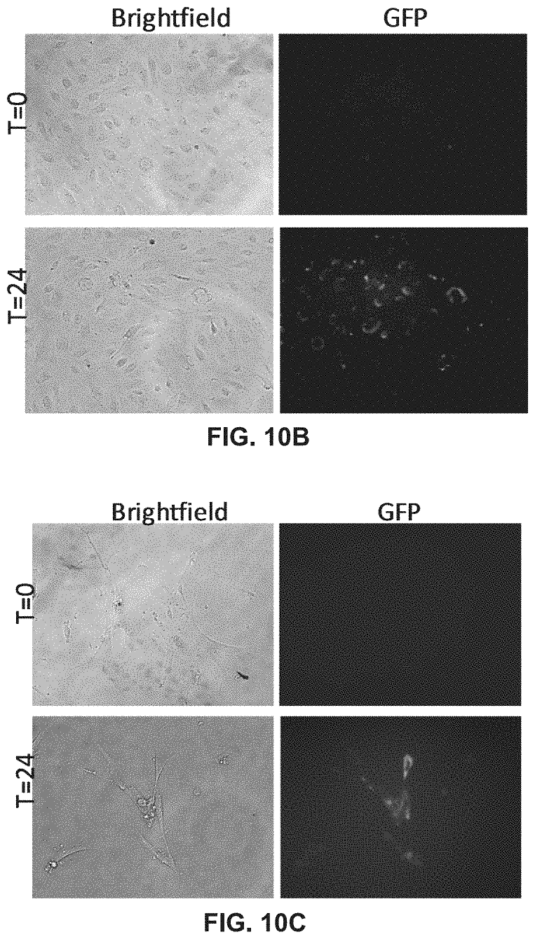

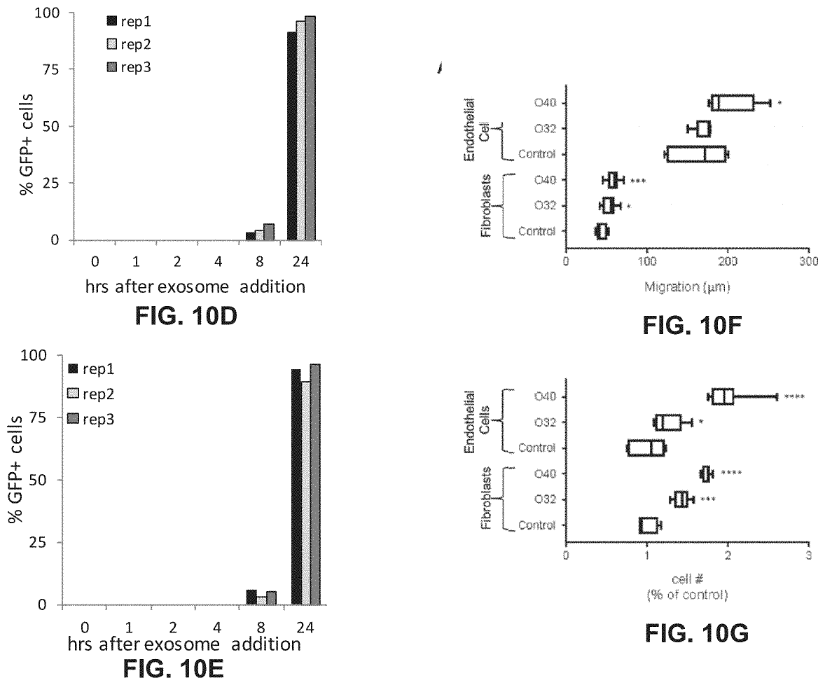

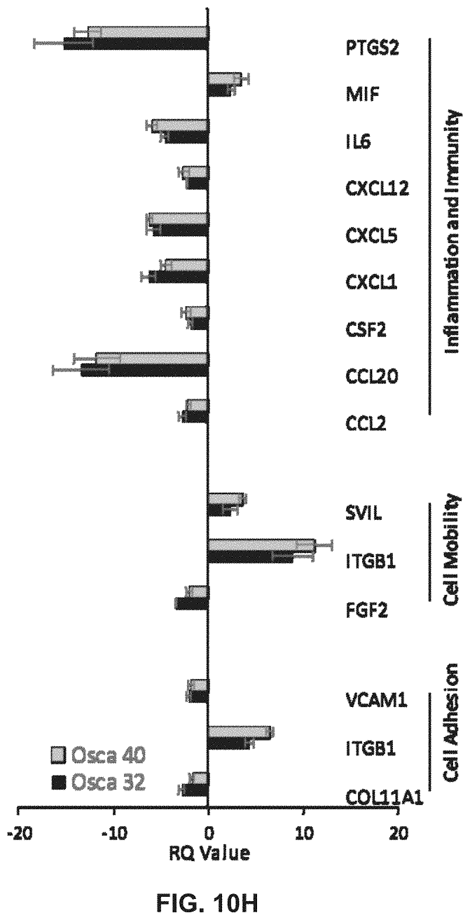

FIGS. 10A-10G illustrate the effects of osteosarcoma exosomes on stromal cells. FIG. 10A is a digital image illustrating osteosarcoma cells transfected with a CD81-GFP fusion construct that is incorporated into exosomes and can be visualized as intracellular and secreted GFP-positive TEX in accordance with examples of this disclosure. As illustrated in FIG. 10A cultured osteosarcoma cells were transfected with CD81-GFP to track synthesis and secretion of exosomes. FIGS. 10B and 10C are brightfield and blue-light images of endothelial cells and fibroblasts, respectively, that had taken up GFP-positive TEX secreted from transfected osteosarcoma cells of FIG. 10A at different times post-TEX introduction in accordance with examples of this disclosure. FIGS. 10B and 10C illustrate osteosarcoma exosomes overlaid on human endothelial cells and fibroblasts, respectively. Internalization of CD81-GFP exosomes was monitored microscopically over 24 hrs. FIG. 10E is a graphical representation of percentages of fibroblast and endothelial cells that had taken up GFP-positive TEX secreted from the transfected osteosarcoma cells of FIG. 10A versus time in accordance with examples of this disclosure. FIG. 10E illustrates quantification of GFP+ cells after exosome addition.

FIGS. 10A-10E illustrate that secreted TEX may be taken up by different cells in a tumor microenvironment. As shown in FIG. 10B and 10E, when GFP-positive TEX was overlaid on cultured fibroblasts or endothelial cells, internalization of GFP-positive TEX was detectable within 6 to 8 hr., with virtually all of the cells showing exosome uptake within 24 hours. TEX derived from different osteosarcoma cell lines had different effects on the transcriptional landscape of target cells (i.e., fibroblasts). Specifically, TEX derived from less aggressive osteosarcoma cells led to decreased expression of transcription factors and increased expression of adhesion molecules by fibroblasts, whereas TEX derived from more aggressive osteosarcoma cells led to increased expression of transcripts associated with IL-17-mediated inflammation and chemotactic factors that attract innate immune cells.

As shown in FIGS. 10A-10E, osteosarcoma-derived exosomes are internalized by stromal cells and influence gene expression. The high propensity for distant metastatic growth in osteosarcoma patients has been well documented and is a key factor in survival rates. The importance of exosomes in promoting a pre-metastatic niche has been characterized in pancreatic cancer as well as melanoma, but the ability of osteosarcoma exosomes to alter the gene signature of target cells has not been fully addressed. To confirm that secreted tumor-derived exosomes could be taken up by stromal target cells, we transfected osteosarcoma cells with CD81 linked to GFP. When overlaid on human pulmonary endothelial cells (FIG. 10B) or on human primary fibroblasts (FIG. 10C), GFP-positive minor-derived exosomes were detectable in target cells within 6 to 8 hours, and virtually all of the target cells had taken up exosomes within 24 hours (FIGS. 10D and 10E).

FIGS. 10F and 10G are graphical representations of migration distance and cell count of different cell lines that had taken up GFP-positive TEX in accordance with examples of this disclosure. FIG. 10F illustrates migration of endothelial cells and fibroblasts in a 2D assay compared to control after exposure to osteosarcoma exosomes from OSCA-32 (O32) or OSCA-40 (O40). FIG. 10G illustrates proliferation of endothelial cells and fibroblasts in a 2D assay compared to control after exposure to osteosarcoma exosomes from OSCA-32 or OSCA-40.

TEX from osteosarcoma cell lines altered the fibroblast transcriptional landscape. TEX from less aggressive cells decreased expression of transcription factors MEF2C, MYOD1, and MYOCD, and increased expression of adhesion molecules. TEX from more aggressive cells increased expression of transcripts associated with IL-17-mediated inflammation and chemotactic factors that attract innate immune cells. As illustrated in FIGS. 10F and 10G, the GFP-positive TEX promoted increased fibroblast and endothelial cell migration and proliferation, as well as altered patterns of kinase signaling in vitro (not shown), which also were dependent on their cell of origin.

FIG. 10H is a graphical representation of qPCR analysis of human fibroblast cells treated with exosomes from OSCA 32 and OSCA 40 cell lines. In the example of FIG. 10H, data shown are genes whose expression changed relative to control by more than 2-fold. These genes may be co-regulated for osteosarcoma.

Previous studies examining osteosarcoma tumor heterogeneity in cell lines indicate they represent the biological behavior of the tumors from which they were originally derived and demonstrated different growth rates and metastatic potential when grown as orthotopic xenografts. Similar to previous findings, tumor derived exosomes from different osteosarcoma cell lines had different effects on fibroblast and endothelial cell migration and proliferation. Exosomes derived from the more aggressive OSCA-40 cell line resulted in an increase in target cell migration and proliferation over control whereas the less aggressive OSCA-32 derived exosomes demonstrated only a slight increase in target cell migration and proliferation over control (FIGS. 10F and 10G). Furthermore, microarray analysis of gene expression of human fibroblasts treated with osteosarcoma-derived exosomes demonstrated altered patterns of gene expression in the categories of cell adhesion, mobility and inflammation, which were also dependent on the exosome cell of origin (FIG. 10H).

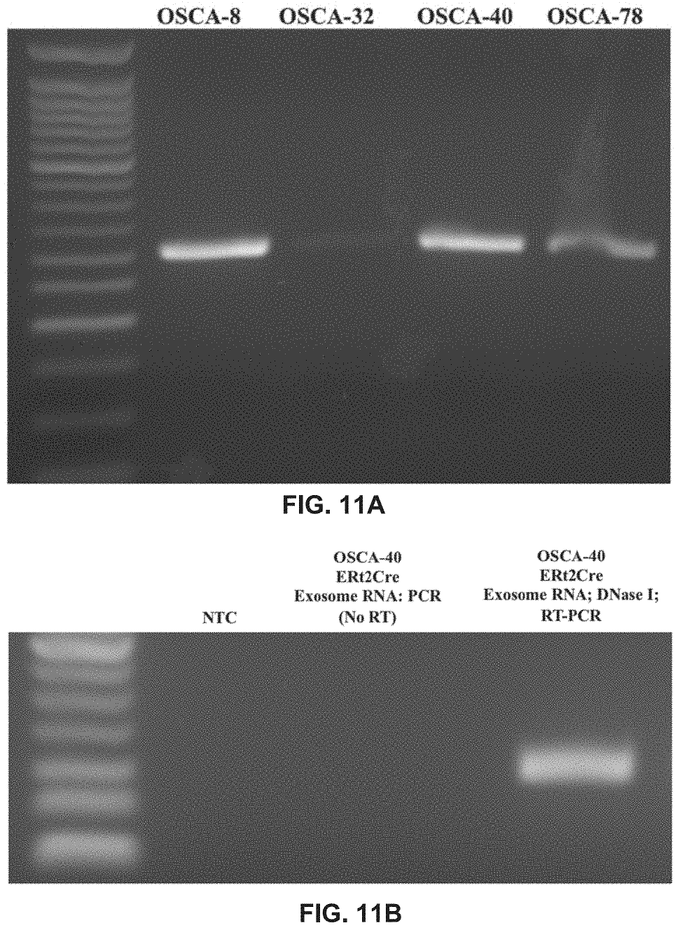

FIGS. 11A and 11B are digital images of results of gel electrophoresis assays illustrating packaging of RNAs from genes transfected or engineered into osteosarcoma cell lines into exosomes by osteosarcoma cells in accordance with examples of this disclosure. FIGS. 11A and 11B illustrate loading of Cre RNA into exosomes in osteosarcoma cells. FIG. HA illustrates results of transfection of a Cre expression vector into dog osteosarcoma cells OSCA-8, OSCA-32, OSCA-40, and OSCA-78. Expression of Cre mRNA in the cells was determined using RT-PCR. Strong bands are present in OSCA-8, OSCA-40, and OSCA-78, and a faint band is present in OSCA-32. FIG. 11B illustrates that exosomes were isolated from OSCA-40 cells, total RNA was isolated, and Cre mRNA was detected by RT-PCR. To control for the potential loading of plasmid DNA, the PCR reaction was done in the absence of RT (left), and DNase I was added to the RNA, prior to synthesis of cDNA (right). Similar data were obtained with the remaining osteosarcoma cell lines.

For example, in addition to intercellular communication, exosomes are used by cells to remove waste and foreign material. Some proportion of genes introduced by transfection or genome engineering thus may be packaged into exosomes. To test this concept, CRE was introduced into osteosarcoma cells. FIGS. 11A and 11B illustrate that Cre mRNA was indeed packaged in exosomes. The same was true using a “super-retinoblastoma tumor suppressor (RB)” construct to reconstitute RB-deficient osteosarcoma cells. This trait may allow the use of genome engineering to introduce CRE into osteosarcoma cells with foxed reporter systems (e.g., cells and mice) to confirm a role for exosomes in intercellular communication. Specifically, a change from red to green in the reporters may indicate that, upon exosome uptake, mRNA in cargo was released, translated to protein (i.e., Cre), and able to edit foxed sites.

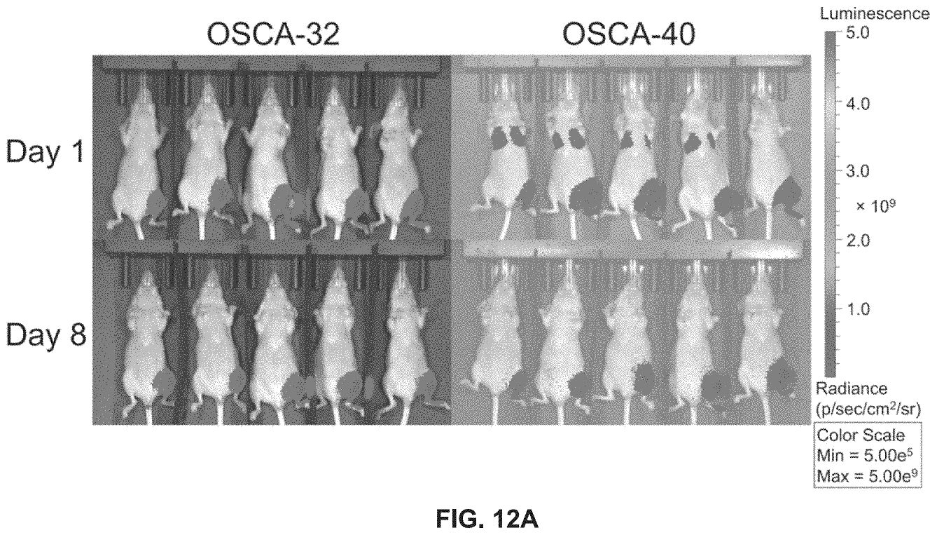

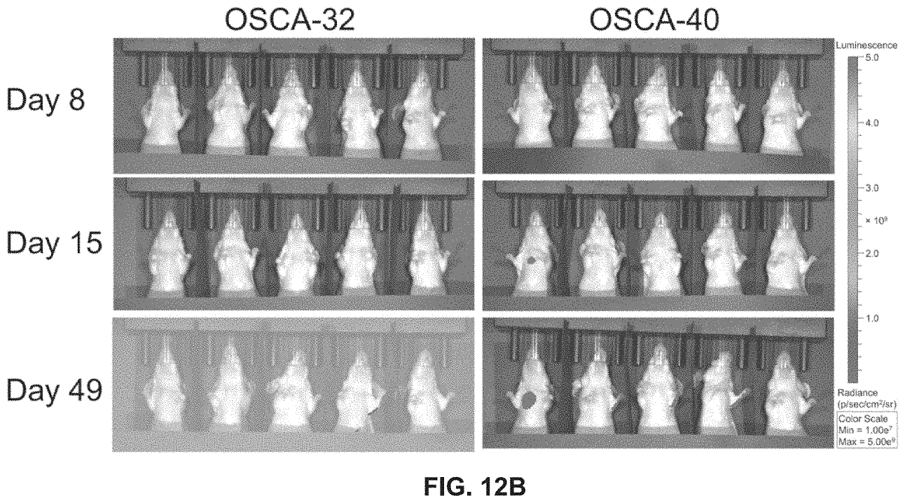

FIGS. 12A and 12B are photographic representations of data pertaining to the application of the techniques described herein with respect to the OS-1/OS-2 xenograft example and illustrating that orthotopic osteosarcoma xenografts may have different metastatic property. Exosomes may be isolated in vivo from serum samples. Methodology to establish orthotopic human and canine osteosarcoma xenografts in mice is described in U.S. Patent Application Publication No. 2018/0105866, which is incorporated in its entirety herein by reference. Osteosarcoma cell lines show stable behavior, recapitulating the characteristics and natural history of the tumor from which they were derived. mRNA cargo in TEX (donor species) may be distinguished from mRNA cargo in the host using the xenografts models by mapping mRNAs to a hybrid genome that allows us to assign reads to the correct species. FIG. 12A illustrates representative luciferase activity at the primary site and lungs of 5 mice at 6 hr. (Day-1) and 1 week after intratibial injection of OSCA-32 and. OSCA-40 cells. OSCA-40 cells transit transiently to the lungs. FIG. 12B illustrates luciferase activity in the lungs of the same mice 1 week, 2 weeks, and 7 weeks after injection. Established metastasis were observed in mouse injected with OSCA-40 cells. At the time of necropsy, approximately 40% of mice injected with OSCA-32 and more than 80% of mice injected with OSCA-40 had evidence of micrometastasis in the lungs.

In the example of FIGS. 12A and 12B, xenografts were established from OSCA-32 and OSCA-40 osteosarcoma cells. Blood was collected from mice serially using facial venipuncture prior to the tumor injections and then weekly for eight weeks. Serum was separated and stored at −86° C. Thawing was done consistently for all samples at 4° C.; samples from each cage were pooled for exosome isolation using the Exoquick method described above. Seramir ExoRNA Amp Kit (SBI) was used for RNA extraction. Approximately 30 ng of RNA could be obtained from exosomes isolated from 250 of pooled mouse serum. Bioanalyzer data indicated that serum exosomes contained mostly small RNAs, but mouse and dog ribosomal RNA (ISS) and mRNA transcripts were detected in these samples using RT-PCR. Sequencing libraries were prepared front exosomal RNA using the SMARTer® Stranded Total RNA-Seq Kit v2—Pico Input Mammalian kit (Clontech). RNA sequencing (50-bp paired-end) was done using a HiSeq 2500 (Illumina); a minimum of 16 million read-pairs were generated for each sample and the average quality scores were above Q30 for all pass filter reads. Analyses of these data to identify relevant biomarkers are described below with respect to preliminary data obtained for Aim 2.

The experimental data below relates to the conceptual approach summarized above with respect to FIG. 3. Experimental details in this section emphasize steps for quality control and quality assurance to maintain rigor and ensure reproducibility.

The cell lines that may be used in this experiment are listed below in Table 1. Each of the cell lines may give rise to tumors in mice when injected orthotopically. The gender imbalance in mice (all four cell lines derived from two females) may be accounted for by using both male and female recipients. Normal human (hfOB), canine (cnOB) and mouse osteoblast cells may be used to control for the effect of cell implantation into the tibiae. Mouse embryo fibroblasts (MEFs) may be generated from Gt(ROSA)26Sor^{tm4(ACTB-tdTomato,-EGFP)Luo}/J (mT/mG) reporter mice to examine Cre activity in vitro. Alternatively, MEFs may be engineered using CRISPR gene editing to insert the reporter. Cell line authentication may be done periodically (e.g., quarterly) for all cells using single tandem repeat markers through IDEXX Bioresearch (MR) IBR reports species of origin, individual cell line authentication, and contamination by all Mycoplasma species known to infect cultured cells.

TABLE 1
Osteosarcoma cell lines that may be used for this experiment

| Cell Line | Species of | | | | Ethnicity, Breed, |
|---|---|---|---|---|---|
| Name | Origin | Sex | Age or Notes from Derivation | | or Strain |
| SAOS-2 | Human | F | 11 | years old | Caucasian |
| U2OS | Human | F | 15 | years old | Caucasian |
| HOS | Human | F | 13 | years old | Caucasian |
| MG-63 | Human | M | 14 | years old | Caucasian |
| OSCA-8 | Dog | M | 1 | year old | Rottweiler |
| OSCA-32 | Dog | F | 9 | years old | Great Pyrenees |
| OSCA-40 | Dog | F | 6 | years old | St. Bernard |
| OSCA-78 | Dog | M | 9.5 | years old | German |
| | | | | | Shepherd Dog |

| | | | | |
|---|---|---|---|---|
| K12 | Mouse | F | derived from a spontaneous tumor | Balb/c |
| | | | of a laboratory mouse | |
| K7M2 | | | subclone selected from K12 cell line | |
| | | | for high metastatic potential | |
| Dunn | Mouse | F | derived from a spontaneous tumor | C3H/HeJ |
| | | | of a laboratory mouse | |
| LM8 | | | subclone selected from Dunn cell | |
| | | | line for high metastatic potential | |

Genome engineering: osteosarcoma cells may be modified to introduce genes encoding a fluorescent protein (CFP) and a bioluminescent protein (firefly luciferase). Independently, the cell lines may also be modified to introduce CRE and genes encoding fluorescent CD81 fusion proteins in the same genomic region. Copy number may be controlled to help ensure reproducibility and comparisons among cell lines.

Effects of genome engineering on exosome contents: RNA may be isolated from unmodified parental cells and from genetically modified cells during the log growth phase of culture and at near-confluency (90%). Exosomes may be collected from cells at the same time for isolation of exosome RNA. Exosome enrichment and RNA isolation procedures and QC may be done as described (also see below). Next generation RNA sequencing (at least 2 million paired end reads, but up to 20 million paired end reads per sample) and routine bioinformatics analysis of transcript abundance may be used to assess differences between parental cells and their genetically modified derivatives, and specifically, potential effects on exosome loading and cargo.

Nude mice may be purchased from an approved laboratory. Nude mice are the strain of choice because they are receptive for osteosarcoma xenografts and allografts and they retain fully functional innate immune systems. Nude reporter mice may be generated through a 3-step breeding strategy using the nude and mT/mG strains in the CS7Bl/6 background. This may allow for growth of Cre-expressing xenografts, which may secrete Cre mRNA in exosomes, enabling tracking of distant effects by change from red to green fluorescence in target organs. Syngeneic, immunocompetent mice may also be used to evaluate the influence of the adaptive immune response on the exosome-associated gene signatures. Balb/c and C3H/HeJ mice may be used, and the data from exosomes generated in these models may be compared to data from the xenografts in Aim 2.

Intercellular delivery of ectopic RNA by exosomes: Cre mRNA expression may be confirmed in genetically modified cells in culture, as well as Cre mRNA loading into secreted exosomes, using RT-PCR. Delivery of functional Cre to target cells may be examined by overlaying Cr e-containing exosomes on mT/mG MEFs, and evaluating changes from red fluorescence to green fluorescence by individual cells. Fluorescent video imaging may be done dynamically over 48 hr., capturing images in the red and green fluorescence channels at 10-minute intervals with the EVOS epifluorescence microscope system.

Orthotopic tumor cell implants: Eight animals per group provide >95% power to identify a 15% change in the median time to tumor when the u for both populations is <2.0 and the acceptable a error is 5% (P<0.05). To account for sex as a variable, equal numbers of male and female e.g., eight male and eight female) mice may be used for the experiments 16 mice per cell line in total). This may also provide a suitable sample size to obtain sufficient blood for exosome isolation and sequencing. Mice may receive buprenorphine for pain control in advance of the procedure and for up to 72 hr. thereafter, as needed. Animals may be assigned to separate cages (e.g., four animals each) in random order, and each cage may receive the same treatment. Intratibial injections (1×10⁵ cells) may be done under general anesthesia and tumor growth may be monitored grossly, comparing the injected tibia to the contralateral tibia, as well as by in vivo imaging. In vivo imaging may be used to monitor development of metastatic disease. The presence of micrometastasis and micrometastasis may be confirmed grossly and microscopically, respectively, as part of the necropsy procedures for each mouse.

Confirmation of TEX release and distant effects on target tissues: Genetically modified tumor cells may be used to confirm that implanted xenograft tumors release exosomes that are taken up by, and that may have a measurable effect on, cells at distant target sites. Cells may be modified to express firefly luciferase. CD81-CFP, and Cre. Nude reporter mice (nu/nu-mT/mG) may be used as hosts. Tumor growth and metastasis may be monitored by luciferase luminescent emission using in vivo imaging. Serum exosomes may be isolated as described herein and quantified using nanoparticle tracking. The proportion of TEX in serum exosomes may be determined by flow cytometry (blue channel) for CD81-CFP. Uptake and biological activity of TEX on target cells at distant sites may be evaluated by changes in the foxed reporter in the lungs, specifically, such as by using the IVIS Spectrum in vivo imaging system.

Blood collection and serum preparation: Blood (100-125 μL) may be collected into containers (e.g., BD microtainer tubes) from all animals in each cage prior to beginning the experiments and then once every two weeks. Sampling may be done by an experienced veterinarian or animal care technician using facial venipuncture in awake to avoid potential effects of anesthesia and to diminish effects of stress from tail vein collection devices. The manufacturer's recommendations may be followed for collection to avoid hemolysis, since free hemoglobin can interfere with RNA isolation and quantification. Blood may be allowed to clot for 30 minutes and serum may be separated by centrifugation and stored for later analysis at −86° C. Hemolysis may be scored for every individual sample, and any sample scoring 1+ or higher may be excluded from the sequencing pools.

Exosome enrichment, RNA isolation, and library preparation for sequencing: The modified procedure for exosome enrichment described above and based on the Exoquick reagent may be used to carry out exosome enrichment. For example, the Seramir ExoRNA Amp Kit may be used for RNA extraction. Commercial kits may be optimized for isolation of small RNAs which are more abundant than mRNAs in exosomes, so for such experiments, the performance of kits available from leading companies may be compared to obtain high quality total RNA, based on yield, size profiles, and amplification of target exosomal mRNAs. Sequencing libraries may be prepared using a validated low-input method, and the quality of each library may be verified before sequencing. Next-generation RNA sequencing may be done at the University of Minnesota Genomics Center (UMGC), with a target of at least 5 million, 50-bp paired-end reads: Routine quality control measures may be done before sequencing data are released for analysis.

Rigor and Reproducibility: Rigor and reproducibility of the techniques and their results described herein may be enabled or enhanced by one or more of the following: cell line authentication protocols; rigorous culture methods; genome engineering and effects on gene expression and exosome loading; mouse breeding, husbandry, and genotyping; validation of exosome release; systemic trafficking and distant effects in vitro and in vivo; statistical power for experiments; xenografts—numbers to account for variability; sample collection protocols—consistent serum preparations (QC); exosome enrichment—nanoparticle tracking and TRPS for quantification and size; immunoblotting; RNA isolation and library preparation; and/or QC for sequencing.

Anticipated Results: Successful production of genomically-edited cells and reporter nude mice may be obtained by the techniques described herein. Exosome loading and cargo may be mostly unaffected by genome engineering. Confirmation of ectopic genes in exosomes and distant effects of Cre in vitro may be obtained. Successful generation of xenografts with predictable behavior (see table for example) may be obtained. Successful isolation of serum exosomes, confirmation of distant effects at targets may be obtained. High quality sequencing data for analysis may be obtained.

TABLE 2
Assumptions:

| | First day to | Proportion with |
|---|---|---|
| Cell line | metastasis | metastasis at end |
| Example OS-1 | Day 7 to Day 10 | About 15/16 |
| Example OS-2 | Day 12 to Day 16 | About 14/16 |
| Example OS-3 | Day 40 to Day 50 | About 8/16 |
| Example OS-4 | >Day 50 | About 1/16 |

Another example aspect described herein is the generation of conserved, exosome-associated mRNA signatures associated with metastatic propensity. TEX cargo has been characterized extensively using cultured tumor cell models. However, it may be unclear if LEX from cultured cells resemble TEX from tumors in vivo, where tumor cells maintain a series of complex relationships with other cells in their local environment and at distant sites. In examples in which TEX are mixed with other host-derived exosomes, some techniques, such as one or more techniques described in U.S. Patent Application Publication No. 2018/0105866, referenced herein in its entirety, may be used to identify the origin of exosome-associated mRNA transcripts from species-mismatched exosomes. Such techniques may enable the establishment of a relationship between TEX cargo in vitro and in vivo and may enable the identification of both TEX- and non-TEX-associated mRNAs that may be used as biomarkers to identify the presence and behavior of a tumor.

Osteosarcoma xenografts were established in nude mice from two distinct cell lines and enriched serum exosomes from mice with and without tumors. Exosomes were collected from tumor-bearing mice and sham-treated controls (e.g., mice injected intratibially with PBS) analyzed, such as by using one or more techniques described in U.S. Patent Application Publication No. 2018/0105866, to catalog TEX-associated mRNAs and host-derived exosome mRNAs. Only sequences that aligned with a single region of the combined reference genome were retained for further analysis to identify DEGS between controls and xenografts. Genes for each species were considered separately for analysis. NGS RNA sequencing was also done for cultured cells to compare TEX mRNA cargo in vitro and in vivo. The mRNA content of serum exosomes from each of the experimental mouse groups before tumor implantation were indistinguishable (no xenograft genes were identifiable). Fifty-one xenograft-derived DEG transcripts (SD>3) were found in TEX, by comparing all transcripts in the tumor groups to all transcripts in the sham group. Only 1.4% of all transcripts found in TEX derived from cultured cells overlapped with TEX-associated transcripts in vivo (39/2,872), so characterization of TEX from isolated tumor cells in culture may not provide a suitable source for biomarker identification. Consistent with the number of differentially expressed, TEX-specific mRNAs in the xenograft experiment, 38 statistically significant, exosome associated host response (mouse) DEGs associated with immune signaling and cellular metabolism were identified when comparing the tumor groups to the sham group.

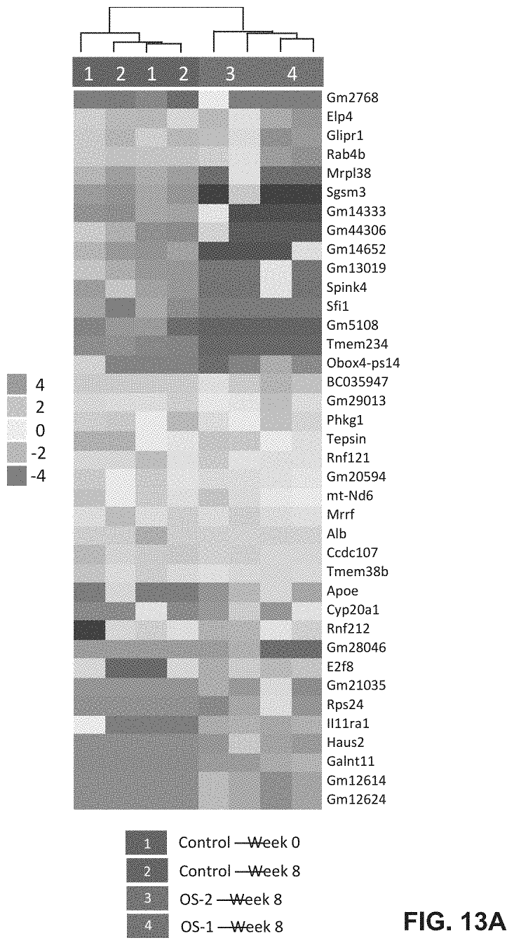

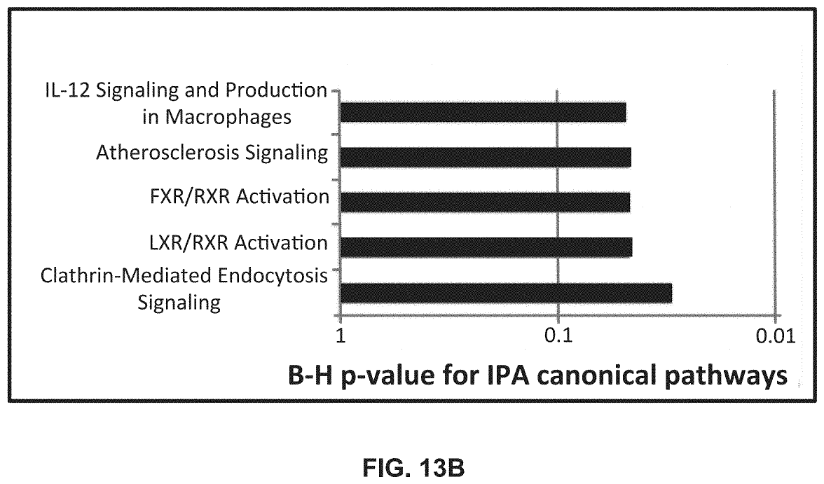

The 38 statistically significant, exosome associated host response (mouse) DEGs and the differential expression thereof across the osteosarcoma groups and control group are illustrated in FIG. 13A, which is a heatmap of the 38 DEG mouse genes. Colored toe bars represent the different experimental samples. The greyscale coded scale represents +/−fold-change in gene expression. FIG. 13B illustrates p-values for IPA canonical pathways associated with functions of one or more of the 38 statistically significant DEGs illustrated in FIG. 13A and identified by IPA as being associated with differentially expressed host (mouse) genes. Collectively FIGS. 13A and 13B illustrate detection of host-specific biomarkers of disease and response to that disease.

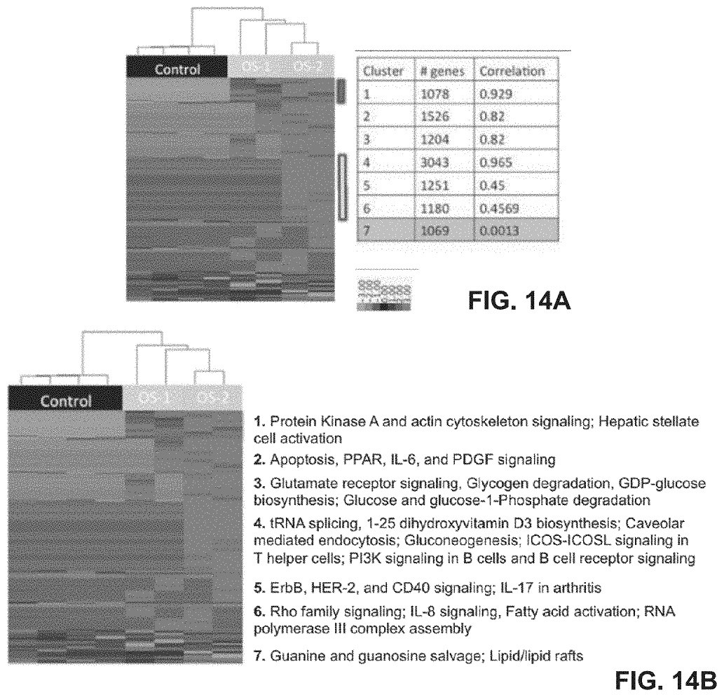

FIGS. 14A and 14B illustrate differentially expressed exosomal mRNA clusters from mice with osteosarcoma xenografts and sham controls. Unsupervised hierarchical clustering of exosomal transcripts from control mice (sham-injected) and from mice harboring xenografts from two different osteosarcoma cell lines with distinct behavior. FIG. 14A is a graphical representation of a data analysis technique in accordance with the examples of this disclosure, as applied to the OS-1/OS-2 xenograft example. Next, significantly associated gene clusters that could distinguish TEX-associated mRNAs were identified from host-derived exosome RNAs. Seven distinct clusters containing between 1,069 and 3,043 differentially expressed mRNAs were identified in the tumor and sham samples, four of which had correlation scores >0.8 as shown in the chart accompanying FIG. 14A. Canonical pathways associated with these clusters included cell signaling, cell death, metabolism, and immune response. Clusters are identified by a color toe bar and by associated functions from IPA. The chart of FIG. 14A indicates number of genes comprising each cluster, as well as their correlation score. Clusters-1 through 6 represent tumor-derived exosomal mRNAs (mapped to donor genome) and cluster-7, which is much more heterogeneous and has a lower correlation score, represents host-derived exosomal mRNAs.

FIG. 14B is a graphical representation of a data analysis technique in accordance with the examples of this disclosure, as applied to the OS-1/OS-2 xenograft example, indicating biological processes and canonical pathways associated with identified gene clusters. FIG. 14B further illustrates a portion of the bioinformatics workflow analysis in accordance with an example technique of this disclosure. As illustrated in FIG. 14B, biological processes and canonical pathways associated with gene clusters identified from the hierarchical clustering heatmap of FIG. 14A may be determined. Top predicted transcriptional regulators of genes for each gene cluster defined by hierarchical clustering are shown in the heatmap and listed alongside the heatmap of FIG. 14B. Cluster 7 of FIG. 14A consists of exclusively mouse genes. ErhB-1 also is named epidermal growth factor receptor (EGFR), and ErbB-2 is also named HER2 in humans and neu in rodents.

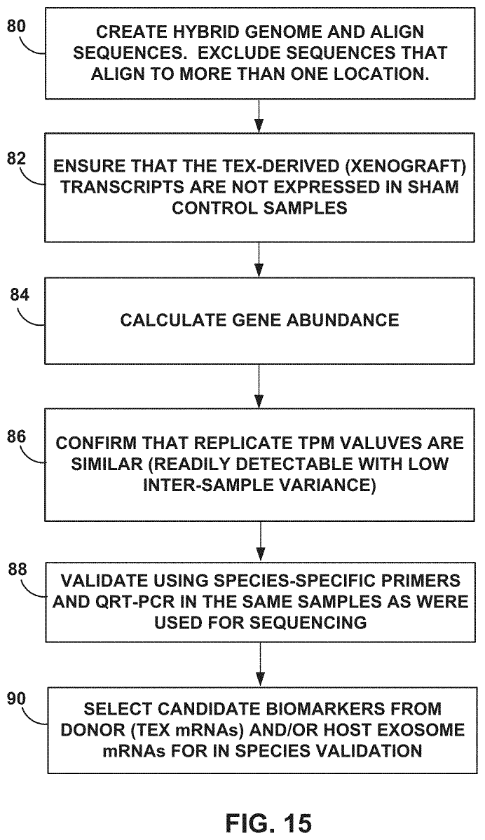

FIG. 15 illustrates an experimental approach to identify TEX-associated transcripts and host exosome-associated transcripts for a particular disease, such as osteosarcoma, FIG. 15 is a flow chart illustrating an example technique for selecting candidate biomarkers for in-species validation of exosomal mRNA signatures, which may inform prognosis of patients with osteosarcoma.

Creation of a hybrid genome and sequence alignment: as illustrated in the example of FIG. 15, hybrid genomes are created with Kallisto index for mapping using the latest donor (human or dog) and host (mouse) genome builds (80). cDNA fasta files are generated that include all donor and host sequences >200 bp to align reads against both genomes. Masked FASTA files may be merged and data may be trimmed, filtered, subjected to FastQC quality control, and insertion size metrics may be calculated for each sample using Picard, which may help ensure that TEX (xenograft transcripts are not expressed in sham control samples (82). Kallisto may be used to generate abundance estimates for each sample (transcripts per million reads, or TPMs) for visualization and analysis (84).

Selection of candidate biomarkers: Data may be analyzed to establish associations between DEGs and GCESS, and tumor growth and time to metastasis by sample, independent of cell line, but restricted by donor species (human with human only and dog with dog only). Correlations between DEGs and GCESS, and time to metastasis may be established by cell line. Overlap between both methods may be defined to establish the most robust predictor biomarkers for biological behavior. Different clusters in each species may achieve maximum power to predict metastatic propensity. Bioinformatically, DESeq2 may be used for differential analysis of transcript counts, converting count values to integer mode, normalizing to library size, estimating dispersions, and calculating log2 fold changes between comparison groups. Genes with a BH adjusted p-value<0.05 and log2 fold change >3 or <−3 between control and xenograft samples may be chosen for initial analysis and validation. Differentially expressed donor genes with DeSeq2 normalized count value greater than zero when mapped to the mouse genome may be removed from the list to avoid confounding. Counts per million (CPM) values may be transformed to log space and mean centered prior to clustering. Data may be analyzed using both unsupervised and supervised methods to identify co-regulated gene clusters (defined statistically by correlation analysis) that are conserved across species and are significantly associated with low or high metastatic propensity. GCESS may be assigned as the sum of the expression value of each gene in the cluster in loge space. Enriched pathway and functional classification analyses of DEGs may be performed using IPA. Gene clusters may be ranked based on the magnitude of difference in expression between groups (more is better), and the inter-sample variance within each group to diminish the effect of outliers (less is better) (86). The top DEGs (biomarker candidates) may be validated in the same samples using qRT-PCR with species-specific primers (88) and selected for in-species validation (90).

Confirmation of gene clusters in syngeneic models and control for immune response: Syngeneic xenografts may be established, for example in the case of osteosarcoma, using K12 and K7M2 cells in Balb/c mice and Dunn and LM8 cell lines in C3H/HeJ. Cells may be modified to express firefly luciferase, CD81-CFP. Serum exosomes may be separated into TEX fractions (CFP-+) and non-TEX fractions (CFP⁻) using flow sorting. RNA extraction and sequencing for TEX and non-TEX cargo may be done as described herein. qRT-PCR may be used to evaluate whether overlapping DEGs selected as candidate biomarkers from the human and canine xenograft models are present in the corresponding mouse exosomes and are predictive for tumor biological behavior. Concomitantly, DEGs may be identified and GCESS analysis may be informed in the syngeneic systems to inform potential contributions from anti-tumor immunity.

Rigor and Reproducibility: Rigor and reproducibility of the techniques and their results described herein may be enabled or enhanced by one or more of the following: bioinformatics controls; setting counts; defining clusters; conservation across species as a means to support objective associations; statistical support of data (probability of error, including multiple testing); and/or reducing multiple testing errors using gene cluster analyses.

Overlapping gene clusters in TEX derived from human and dog osteosarcoma associated with time to metastasis in mice. Confirmation of presence for these genes in syngeneic mouse osteosarcoma. Conserved host response in xenografts, with overlapping elements in syngeneic mouse model, with notable appearance of mRNAs in host response exosomes associated with immune function (T cells). Refinement of gene list and clusters to create a biomarker set for in species validation in dogs and humans (e.g., overlapping elements and species-specific elements).

The example experiment described herein may validate the exosome mRNA signature in an annotated cohort of samples from children with osteosarcoma to establish its predictive value. Biomarkers to predict biological behavior and the likelihood of metastatic progression are an unmet need for osteosarcoma patients. Such biomarkers may enable personalization and/or improvement of treatment efficacy, and/or may reduce acute or chronic therapy-related side effects. Xenografts may provide a powerful model to identify TEX-associated and non-TEX (host response)-associated mRNAs for use as biomarkers of disease progression. As discussed below, the diversity of cell lines used to model the xenografts may provide a sufficiently large representation of the heterogeneity observed across tumor patients, which may enable identification of relevant biomarkers associated with tumor behavior, and may enable prediction of time to metastasis. As further discussed below, the filter provided by species-specific selection of mRNA biomarkers of disease and host response may reduce the number of samples needed for discovery of relevant, diagnostically useful biomarkers considerably, perhaps from thousands to dozens or a few hundred. Such a reduction in the number of samples needed may be important in the case of rare diseases such as osteosarcoma, where amassing hundreds of samples requires years and heavily coordinated participation from multiple institutions.

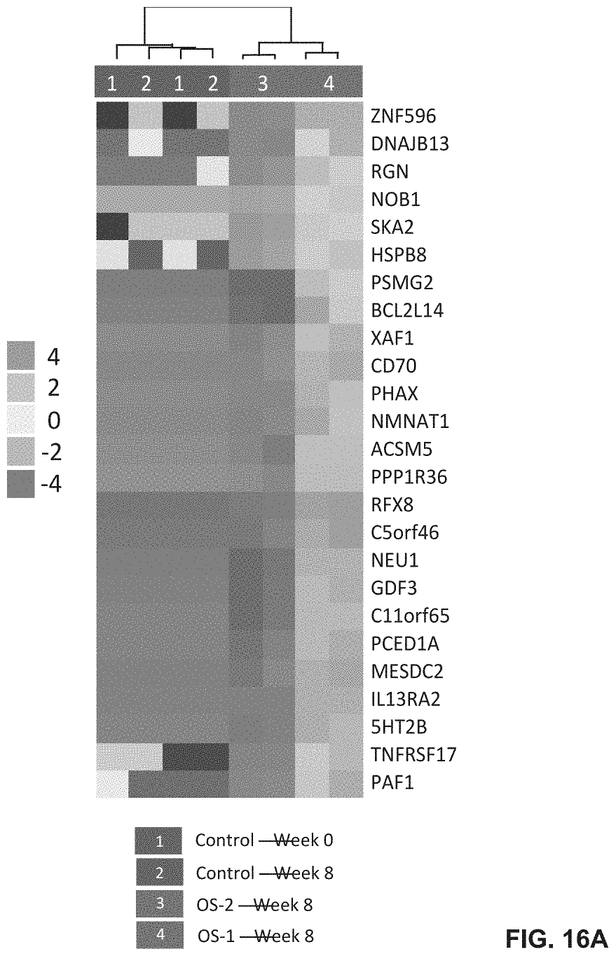

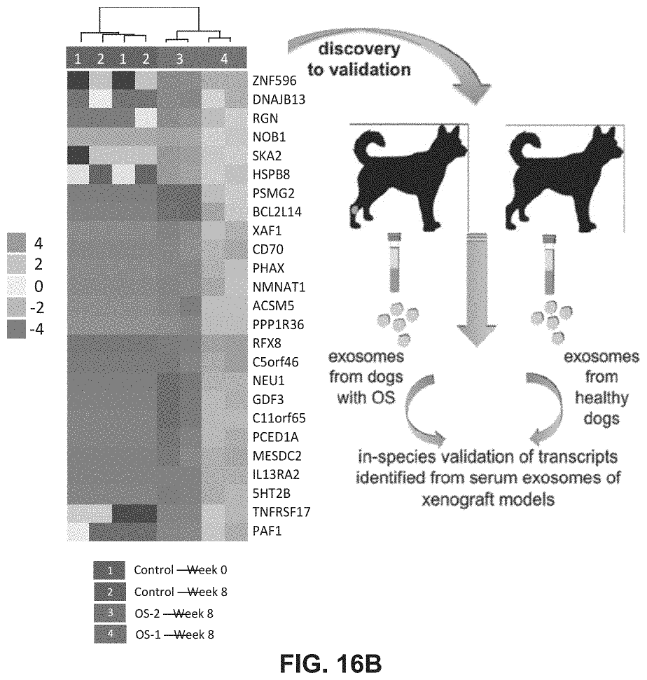

The techniques described herein with respect to determining the exosome mRNA signature may validate these assumptions in samples from individuals of a target species with a corresponding disease, as discussed below with respect to the preliminary data represented in FIGS. 16A-21. Taken together, the techniques described herein (e.g., as applied in the examples of FIGS. 16A-21) may enable robust identification of cell-free exosomal nucleic acid biomarkers combined with machine learning for biological status determination, such as to determine a likelihood that an organism may develop a particular disease, has a particular disease, or another biological status of interest. In some examples, such techniques may be applied with serum-derived biomarkers in non-human animal patient or human patient samples for clinical utility.

FIG. 16A is a graphical representation of the detection of biomarkers of disease and host response, as applied to the OS-1/OS-2 xenograft example, and illustrates target genes and schematic for validation. The heatmap of FIG. 15A illustrates an example 25 most differentially expressed, coordinately regulated canine transcripts from exosomes identified by statistical testing with ‘DESeq2’ from the osteosarcoma xenografts. The heatmap of FIG. 16A illustrates an intensity of color that is relevant that that specific gene which has been normalized to the mean or median for the group. However, the change in greyscale intensity (e.g., from the left columns to the right columns) indicate the change in direction of expression (e.g, up or down expression) that can be used to determine coordinate regulation or co-regulation of the genes.

These 25 gene transcripts may be referred to as a group of osteosarcoma-linked genes. The group of genes may be coordinately regulated because they are typically turned on and off together as a result of the presence of osteosarcoma. These 25 genes include ZNF595, DNAJB13, RGN, NOB1, SKA2, HSPB8, PSMG2, BCL2L14, XAF1, CD70, PHAX NMNAT1, ACSM5, PPP1R36, RFX8, C5orf46, NEU1, GDF3, C11orf65, PCED1A, MESDC2, IL13RA2, 5HT2B, TNFRSF17, and PAF1. In some examples, the group of osteosarcoma-linked genes may include fewer, greater, or different genes than the example genes described in FIG. 16A. Therefore, the gene signature may include a plurality of any of the 25 genes identified in the group of osteosarcoma-linked genes. In one example, the gene signature may include at least five genes selected from the group of osteosarcoma-linked genes. In some examples, as few as four genes may enable a gene signature providing sufficient detection of the target disease, such as osteosarcoma. In some examples, 6, 7, 8, or more genes may be used as part of the gene signature. Additional genes may increase the specificity of the diagnosis in some examples. However, in some examples, as few as 2, 3, or 4 genes may be acceptable to form the gene signature. In one example, the gene signature may include five genes, and may include SKA, NEU1, PAF1, PSMG2, and NOB1.

Next-generation sequencing of orthotopic xenograft exosomal mRNAs identifies species-specific differentially expressed genes. Tumor derived exosome cargo has been characterized extensively using cultured tumor cell models. However, it is unclear if these studies are directly translatable to in vivo studies where tumor cells maintain a series of complex relationships with other cells in their local environment and at distant sites. One of the challenges that has made this comparison difficult is that tumor-derived exosomes are mixed with other host exosomes. To circumvent this problem, we used orthotopic xenograft models, where we were able to distinguish tumor-derived exosomal mRNAs from host exosomal mRNA bioinformaticaly. Briefly, xenografts in nude mice were established using two osteosarcoma cell lines with different biological behavior, collected serum exosomes from tumor-bearing mice and sham-treated controls, and performed next-generation sequencing to characterize the full complement of exosomal mRNAs. Sequences were aligned to a hybrid genome of mouse and canine genes. Only sequences that aligned with a single region of the combined reference genome were retained for further analysis to identify differentially expressed genes between controls and xenografts. The mRNA content of serum exosomes from each of the experimental mouse groups before tumor implantation were indistinguishable (no xenograft genes were identifiable). As discussed above, thirty-eight differentially expressed genes (DEGs) were specifically associated with the host response (mouse) and 51 canine specific genes were reproducibly identified in the exosome samples from mice harboring canine osteosarcoma xenografts, of which the 25 most differentially expressed and found at consistently high levels were selected (FIGS. 13A and 16A). It may be beneficial to focus on the most differentially expressed dog genes to define biomarkers that could indicate the presence of osteosarcoma in dogs. This list was further narrowed to 10 genes with significantly different expression (SD>3) that were highly expressed in both tumor groups, but not in the sham controls. These species-specific primers were validated for linearity and specificity for each gene and confirmed that the genes were detectable individually in serum exosomes from canine osteosarcoma patients. This resulted in a final list of five genes that were used for in-species validation.

The process for validation of these genes from FIG. 16A in samples from dogs with osteosarcoma is illustrated in FIG. 16B. Canine-specific cluster-I genes were analyzed for further validation to define a set of biomarkers that could establish the presence of osteosarcoma in dogs. The candidate list was narrowed to 10 known genes that showed significantly different expression (SD>3) and also were highly expressed in both tumor groups (i.e., were always present in animals with tumors and would be feasible to detect by RT-PCR), but not in the sham controls, and that had small inter-sample variance. The species-specific PCR primers (donor and host) then were validated for linearity and specificity for each gene, which confirmed that the genes were detectable individually in serum exosomes and that all originated from the xenograft donor. This resulted in a list of five candidate gene markers that were carried to the in species validation as described below.

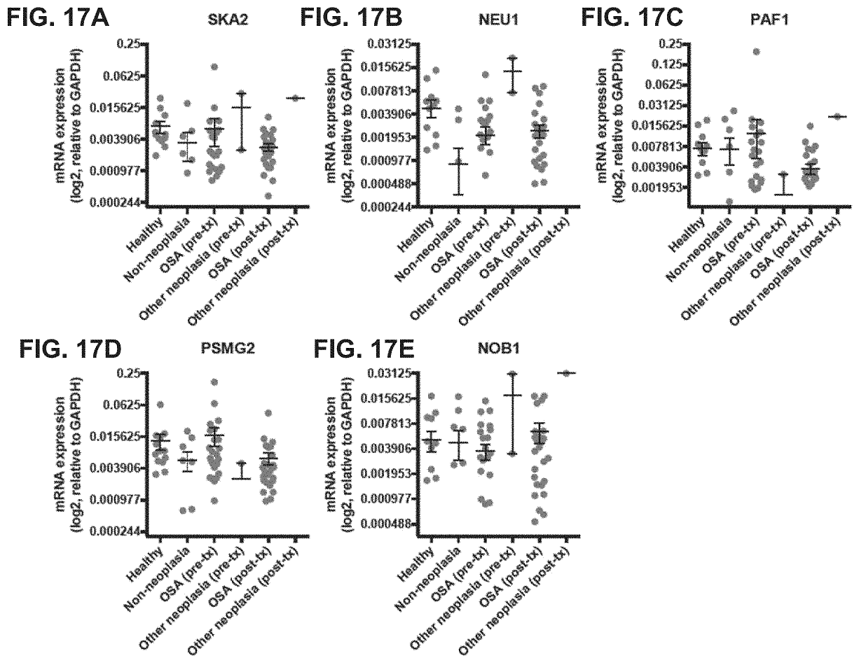

Preliminary Data: In species validation of the mRNA signature identified in canine osteosarcoma xenografts was done in a cohort of archival samples from the University of Minnesota and The Ohio State University encompassing 53 dogs. FIGS. 17A-17E are biomarker expression profiles of messenger RNA (mRNA) associated with each of five genes of an identified gene signature associated with osteosarcoma for each of the 53 dogs.

The cohort of 53 dogs included a group of 28 dogs with osteosarcoma, from which blood samples were obtained at diagnosis (pre-treatment, n=26) and after treatment (amputation+/−chemotherapy, n=27) at various timepoints ranging from 2 to 984 days with a median of 37 days post-treatment. A group of ten dogs had non-neoplastic diseases, and a group of two dogs had tumors of bone that were different from osteosarcoma, including one for which there were pre-treatment and post-treatment samples. A group of thirteen dogs were healthy, with no apparent disease. Enrichment of serum exosomes and RNA isolation were done as described above for mouse samples.

qRT-PCR was used to amplify five candidate biomarkers, SKA2, NEU1, PAF1, PSMG2, and NOB1 and expression values were normalized to GAPDH using the ΔCq method. The normalized expression values are illustrated in FIGS. 17A-17E for respective ones of SKA2, NEU1, PAF1, PSMG2, and NOB1. As illustrated in FIGS. 17A-17E and as further discussed below, expression levels for each of the five candidate biomarkers varied among the groups of the cohort of 53 dogs. It is noted that other qualitative methods for determining the abundance of mRNA may be used. For example, a direct RNA detection technique with multiplexing, such as QuantiGene, may be employed for verification and detection of serum or exosomal RNAs as replacement for qRT-PCR. A 96-well plate luminometer or Luminex machine can be used for multiplexing 3 to 80 RNA targets within one sample (with bead-based detection), without the need for RNA extraction and purification.

FIG. 18A is a graphical representation of results of principal component analysis of the plurality of samples of FIGS. 17A-17E, and FIG. 18B is a graphical representation of results of linear discriminant analysis (LDA) of the plurality of samples of FIGS. 17A-17E.

FIGS. 18A and 18B include data visualization and machine learning performance using pre-treatment (pre-tx) samples as dataset for training and developing machine learning models. As shown in FIG. 18A, principal component analysis (PCA) scatter plot of data shows principal components (PCs) 1 vs. 2, 1 vs. 3, and 2 vs. 3. FIG. 18B illustrates the three-component linear discriminant analysis (LDA) scatter plot of data shows LDA components (LDs) 1 vs. 2, 1 vs. 3, and 2 vs. 3. While neither method of FIGS. 18A or 18B achieved robust separation of the four groups using the 5-gene signature, the data appeared amenable to training using machine learning.

Relative expression data were mean centered and scaled based on the standard deviation across all samples for each gene prior to performing PCA, PC1, PC2, and PC3 identified on respective ones of the X, Y, and Z axes of FIG. 17E are principal components 2, and 3. Post treatment samples were excluded from this example analysis, but could be included in other examples. Unique patterns of expression that differentiated dogs with osteosarcoma from healthy dogs and dogs with non-neoplastic conditions were identified using both PCA and LDA. Specifically, osteosarcoma samples showed tight clustering in both PCA and LDA, while healthy and non-neoplasia samples were more spread out.

Machine learning models predict osteosarcoma. To minimize bias in machine learning, 3-component LDA-transformed data was used from 52 “no apparent disease”, “non-neoplasia”, “pre-treatment osteosarcoma”, and “other neoplasia” samples as the training set for 12 independent artificial intelligence algorithms. Top performing models were further tested on the training set using 10-fold cross-validations with 100 iterations. FIG. 18C is a graph of example averaged machine learning performance comparison across four selected models. Data shown as mean±SD (n=10 repeats). Sensitivity: probability of selecting true Osteosarcoma; specificity: probability of selecting non-Osteosarcoma (Healthy, Other neoplasia, and Non-neoplasia). As shown in FIG. 18C, four high-performing models based on accuracy, sensitivity and specificity were: k-nearest neighbor (KNN), BAG, random forest (RF), and EXT, with sensitivity and specificity of approximately 75% on average.

According to these machine learning techniques, quantified transcripts from each sample may be applied to one or more machine learning models to classify the sample as one of the plurality of possible classifications or biological status (e.g., healthy, non-neoplasia, osteosarcoma, other neoplasia, or the likelihood of having or not having a certain condition). In some examples, the system may associate the biological status of the subject with the corresponding pattern of expression (e.g., the quantified mRNA abundance) of the of the plurality of gene sequences by applying the pattern of expression to respective machine learning models of the one or more machine learning models. The system may also then determine that the machine learning models of the one or more machine learning models converge on the biological status front a plurality of biological statuses. In this manner, the system may only determine a biological status for a sample when multiple machine learning models give the sample the same classification (e.g., converge on the biological status).

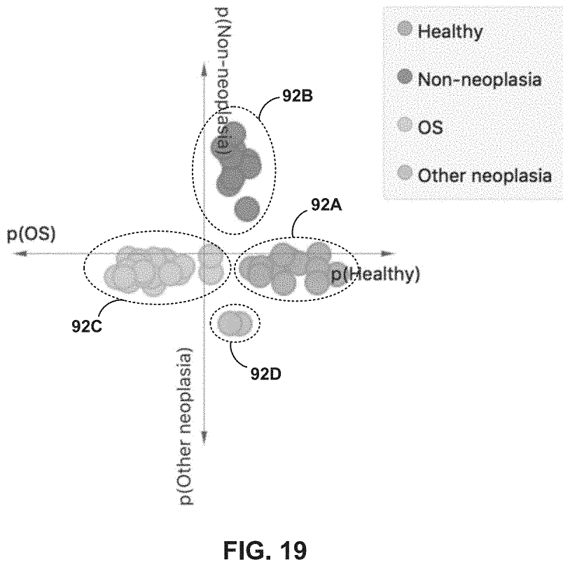

FIGS. 19-21B illustrate that the exosomal five-gene signature of SKA2, NEU1, PAF1, PSMG2, and NOB1 described above with respect to the tumor xenograft model may be used in a machine learning environment to define the presence of osteosarcoma in dogs.

To illustrate the use of the five-gene signature in a machine learning environment to define the presence of osteosarcoma in dogs, a supervised classification strategy using machine learning algorithms was used, including support vector machine (SVM), k-nearest neighbors (kNN), random forest (RF), neural network (NN), and. CN2 rule inducer (CN2). Fifty-two samples from groups of dogs that included healthy dogs, non-neoplasia dogs, pre-treatment osteosarcoma dogs, and dogs having other neoplasia were used as the training set, and the remaining 28 post-treatment samples were treated as unknowns and used as the test set. As discussed below, RF, NN, and CN2 machine learning models produced high classification accuracy that would provide diagnostically useful information when tested against the training data set, suggesting that the canine expression data could be trained using these three algorithms.

FIG. 19 is probability plot of results of predicted classifications of a set of samples using the CN2 rule inducer model. In the example of FIG. 19, only pre-treatment data were used. In FIG. 19, group 92A represents healthy dogs for which treatment status is not applicable. Group 92B represents non-neoplasia dogs for which treatment status is not applicable. Group 92C represents pre-treatment (pre-tx) OSA dogs. Group 92D represents two pre-tx other-neoplasia dogs. Two of 27 osteosarcoma samples fell within the center of the axes. The one on top had no detectable expression in all five genes, thus cannot be properly trained and may result in an ambiguous prediction. The one point on the bottom near the center axes may also be ambiguous, but is closer to the other neoplasia category.

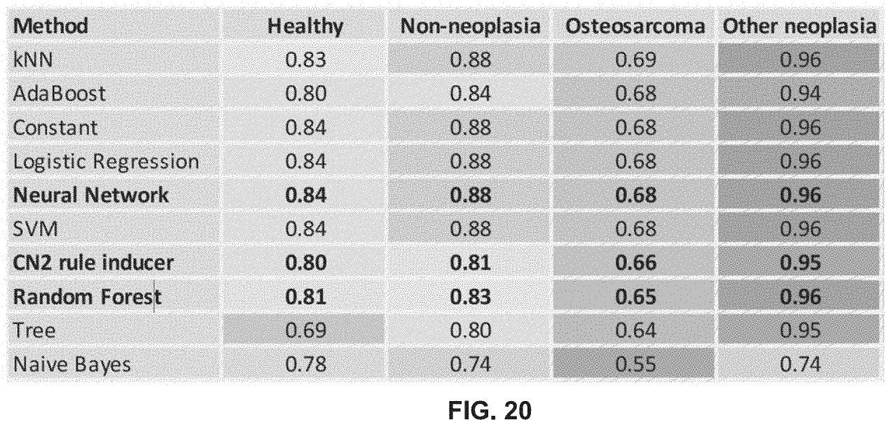

FIG. 20 is a chart illustrating classification accuracy of a set of samples based on cross-validation analysis with different machine learning models. Classification accuracy (shown as ratio) of all 80 samples based on 10-fold cross-validation analysis with the different machine learning methods. In sonic examples, a use of multiple machine learning models to determine a biological status of an organism may provide one or more benefits relative to the use of a single machine learning model. For example, using multiple machine learning models may enable comparison of results obtained from each of the multiple models for a sample from art organism to identify which model provides the best fit of the data (e.g., the expression levels of the different genes of the gene signature of interest) derived from the RNA sequences of the sample to the known expression patterns associated with known biological statuses that correspond to the disease or other condition of interest and highest classification accuracy of the biological status. For example, if the machine learning models support an assumption (i.e., multiple models generate equivalent results), this may provide greater assurance that the data are trainable—in other words, that test results will be reliable.

In examples in which one or more of multiple models provides a better fit for data derived from the sample, that model may enable a more accurate determination of the biological status of the organism than models that do not provide as good of a fit of the data. For example, processing circuitry of a computer system (e.g., processing circuitry 46 of computing device 42) may determine patterns of gene expression associated with a sample and analyze such patterns using each of a plurality of machine learning models. The processing circuitry then may select the model that provides the best fit of the data from the sample and determine the biological status of the organism based on a biological status associated with the gene expression patterns from the model that best fits the data. In some examples, the same machine learning models may not provide the best accuracy for each sample (e.g., in examples in which different gene signatures and/or diseases of interest are used). Thus, the ability to analyze data from a sample by fitting data using multiple machine learning models may increase the robustness and accuracy of these techniques across multiple applications.

In the experiment described above classifying 27 dogs, the final diagnosis of each subject and the post treatment (post-tx) sample classification using the prediction algorithm described herein ex:., using machine learning models) compared to the number of days until each subject relapsed to a type of cancer (e.g., Osteosarcoma (OSA) or non-osteosarcoma (non-OSA)). The 27 post-treatment samples were then as “unknowns” to implement our machine learning models as a means to detect the presence of osteosarcoma (i.e., residual disease). The data are presented in Table 3 below, showing 61% (KNN), 64% (BAG), 68% (RF), and 64% (EXT) of the samples were classified as osteosarcoma. Using a binary assignment of “osteosarcoma” or “non-osteosarcoma,” 14 of the 27 samples were classified as non-osteosarcoma. We surmised this could be indicative of changes in disease state following treatment, reflecting presence or absence of molecular residual disease that could predict overall survival. Survival data available for a subset of nine dogs that had received uniform therapy, and for which samples had been obtained at the same time after treatment. Kaplan Meier survival probability analysis for those nine dogs showed that the dogs identified as having osteosarcoma present post-treatment by all four learning models had shorter overall survival times (Chi square value=2.99, p=0.08) than those post-treatment dogs that were identified as not having osteosarcoma present (i.e., mixed prediction calls).

TABLE 3

| | | Post-tx | Days till |
|---|---|---|---|
| Sample | Diagnosis | Classification | relapse |
| Dog 01 | OSA | Non-OSA | 365 |
| Dog 04 | Other neoplasia | Non-OSA | n/a |
| Dog 07 | OSA | OSA | 244 |
| Dog 08 | OSA | Non-OSA | n/a |
| Dog 10 | OSA | Non-OSA | n/a |
| Dog 11 | OSA | Non-OSA | 74 |
| Dog 13 | OSA | Non-OSA | 456 |
| Dog 14 | OSA | OSA | 102 |
| Dog 15 | OSA | Non-OSA | 189 |
| Dog 16 | OSA | Non-OSA | 284 |
| Dog 17 | OSA | OSA | n/a |
| Dog 20 | OSA | Non-OSA | n/a |
| Dog 23 | OSA | Non-OSA | n/a |
| Dog 26 | OSA | Non-OSA | n/a |
| Dog 29 | OSA | Non-OSA | n/a |
| Dog 32 | OSA | Non-OSA | n/a |
| Dog 35 | OSA | OSA | 60 |
| Dog 36 | OSA | OSA | 179 |
| Dog 37 | OSA | OSA | n/a |
| Dog 38 | OSA | OSA | n/a |
| Dog 39 | OSA | OSA | n/a |
| Dog 42 | OSA | OSA | n/a |
| Dog 45 | OSA | OSA | n/a |
| Dog 48 | OSA | Non-OSA | n/a |
| Dog 49 | OSA | OSA | n/a |
| Dog 50 | OSA | OSA | n/a |
| Dog 53 | OSA | OSA | n/a |

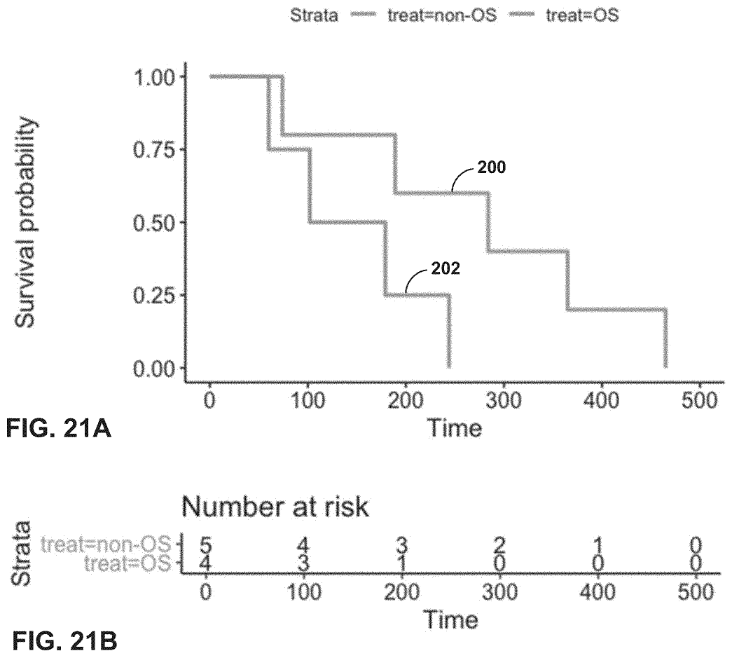

FIGS. 21A and 21B are graphical representations of survival probability of subjects classified using the machine learning models. As shown in FIG. 21A, the graph indicates example of overall survival probability over time between subjects classified as osteosarcoma or non-osteosarcoma. The Kaplan-Meier survival curves 200 and 202 demonstrate time to relapse for subset of canine osteosarcoma patients with available survival data, comparing those whose post-treatment samples were classified as “osteosarcoma” (curve 202) with those whose post-treatment samples were classified as “non-osteosarcoma” (curve 200). The data used to derive curves 200 and 202 is from the dog subjects from Table 3. The classifications correctly predicted that osteosarcoma classified dogs had lower survival rates than the dogs classified as not having osteosarcoma. FIG. 21B shows the actual number of animals at which relapse occurred for each group.

In the above discussed study a platform to identify mRNAs in tumor derived exosomes as biomarkers of disease and identified a gene signature that was utilized in machine learning models to correctly predict osteosarcoma with approximately 75% sensitivity and specificity was developed. Through the use xenografts, species-mismatched exosomes were generated in a single environment; the combined host and donor exosomes were isolated using well-established methods, prepped for next generation sequencing and a custom bioinformatics pipeline was applied. Taking advantage of the xenograft system allowed us to successfully identify donor- and host-derived exosome mRNAs and from this sequencing data, a five-gene signature was identified to predict osteosarcoma in canine patient samples.

The sequencing results from the orthotopic xenograft exosomal mRNAs revealed 38 statistically significant differentially expressed genes that were specifically associated with the host response and 25 highly differentially expressed, statistically significant genes that were indicative of the presence of canine osteosarcoma cells in the mice. The differentially expressed host genes were primarily associated with immune signaling and cellular metabolism, consistent with previous reports of immune system involvement with cancer progression. Although beyond the scope of this study, these data support the idea that our xenograft approach may also have the capability of identifying potential biomarkers of the host response. Future studies focusing on this population of genes and how they change in relation to tumor growth and metastasis would be beneficial in identifying biomarkers that can define host response to tumor, as well as the host response to therapy.

To establish proof of concept, the most differentially-expressed dog genes were used to define biomarkers that could establish the presence of osteosarcoma in dogs and narrowed the list of 25 to the following five genes: SKA2, NEU1, PAF1, PSMG2 and NOB1. However, other genes may be used in other examples. Interestingly, routine qRT-PCR results for individual genes from exosome mRNA isolated from archived serum samples of 53 dogs did not reveal significant changes in expression across our various cohorts. This data are in agreement with reports that univariate approaches are not the optimal method for identifying biomarkers from large data-sets, as they tend to ignore gene interactions. The data also support the concept that multivariate selection methods should be applied for the discovery of robust biomarkers. To test this theory that coordinated expression of all five genes could predict the presence of osteosarcoma (i.e., detectable disease burden), statistical transformations of the data were performed using principle component analysis as well as linear discriminant analysis. This approach allowed us to observed a slight separation that could discriminate dogs with osteosarcoma from healthy dogs and dogs with non-neoplastic conditions. This discrimination was more evident with LDA, which also allowed observation differences in gene expression between dogs with osteosarcoma in the pre-treatment group and in the post-treatment group.

To leverage this observed separation, machine learning models were used to predict the presence of osteosarcoma. The top performing model was KNN, which iterated 10-fold cross-validation analysis showed a recall and f1-score of 0.83 and 0.70, respectively. The prediction summaries for the post-treatment samples indicated a similar, but slightly lower percentage of osteosarcoma samples identified using machine learning. We attributed this discrepancy to the lower limit of detection of the assay for minimal residual disease. In other words, dogs classified as “non-osteosarcoma” would be considered to be in molecular remission. This possibility was addressed by evaluating the lag time to relapse in a subset of dogs classified as having osteosarcoma, or not having osteosarcoma, that had received the same treatment and been tested at the same timepoint in the course of their therapy. The results from this analysis were consistent with the hypothesis: dogs were identified as having osteosarcoma post-treatment had shorter overall survival than post-treatment dogs that were classified as non-osteosarcoma.

Some limitations of this study include the number of primary samples available, incomplete metadata for some samples, and the small number of mice and osteosarcoma cell lines used in the xenograft biomarker identification experiments. Additional mice may be helpful to expand the list of biomarkers, as well as dynamic changes over the course of disease. Similarly, methods encompassing greater numbers of biomarkers in the assays, and tracking these biomarkers in prospective, controlled cohorts, will improve the resolution, precision, sensitivity, specificity, and predictive value of the tests, allowing them to be used both is the early detection setting (for canine osteosarcoma risk), as well as to monitor molecular remission and provide the option to implement rescue therapies in advance of clinical relapse. These results provide proof of concept for a xenograft platform that can identify cancer biomarkers, with in species validation for presence of osteosarcoma in dogs. The results also document the implementation of machine learning to leverage such data into clinically useful tests to inform risk assessment and prognosis.

The following is an example experimental approach for identifying biomarkers for disease for humans. First, osteosarcoma samples may be obtained for validation. A number of de-identified serum samples from children with osteosarcoma may be obtained (e.g., from the COG biorepository). Samples may be coded, and may include metadata for age, sex, ethnicity, disease stage, and temporal relationship between diagnosis and when the sample was acquired. De-identified serum samples from dogs with osteosarcoma have been obtained from the Pfizer CCOGC biorepository. Samples include metadata for age, sex, breed, disease stage, arid temporal relationship between diagnosis and when the sample was acquired. Both of these groups have national sample collection efforts that are conducted under strict SOPs. Sample storage is consistent, and quality control and quality assurance are well documented. Samples are annotated with follow up information and outcome. Samples may be assigned to relevant groups for analysis according to time to metastasis and time to death. Samples that cannot be assigned to an outcome event may be censored for analysis. Enrichment of serum exosomes and RNA isolation may be done as described in Aim 2.

Statistical planning and power: Analysis groups for children may include (1) Yes/No metastasis at diagnosis; (2) Yes/No risk for relapse post-treatment (time to metastasis less or more than 5 years); and (3) Yes/No death event (overall survival at 10 years). For dogs, the timelines for relapse and survival will be adjusted accordingly (8 months and 2 years, respectively). Assuming equal numbers of samples, 31 samples per group may have 80% power, and 41 samples may have 90% power, to detect a difference of 0.20 in the area under the ROC curve (AUC) under the null hypothesis of 0.50, and an AUC under the alternative hypothesis of 0.70 using a two-sided z-test at a significance level of 0.05. The data are continuous responses. The AUC is computed between false positive rates of 0.00 and 1.00. The ratio of the standard deviation of the responses in the negative group to the standard deviation of the responses in the positive group is 1.00. Estimates for sensitivity and specificity are expected to have confidence interval widths of 0.118 for n=31 and 0.108 for n=41, when the expected sensitivity and specificity are at least 80%. If 100 samples represented 70 highly aggressive tumors and 30 less aggressive tumors, sensitivity and specificity could be estimated with confidence intervals (CI) of 12% and 19%, or less. In example having 100 samples in each group, both sensitivity and specificity may be estimated with CI of <10%.

Quantification of gene expression: NanoString technique may be used to quantify gene expression, since it has high tolerance for degraded RNA and a larger number of genes in the clusters can be tested efficiently. Furthermore. NanoString offers custom design for human and canine qNPA arrays. Quantification of transcript abundance using NanoString or qRT-PCR may include gene sequences used for calibration and normalization and may be done following FDA guidance. Outcome data may be blinded until all RNA data are collected and tabulated. Relationships between exosomal mRNA signatures and patient outcomes may be analyzed using unsupervised methods, including principal components analysis (PCA), and supervised linear discriminant analysis (LDA). Iterative training and validation may be used for machine learning algorithms. Ten percent of samples may be randomly assigned to serve as technical replicates in qNPA, and a different 10% of samples may be used to quantify gene expression by qRT-PCR, normalized against three independent housekeeping genes. If discrepancies arise between the two methods, cross-validation may be increased, such as up to about 25% of samples.

Data analysis: Relationships between gene clusters and patient outcomes may be analyzed in humans and in dogs or other non-human animals using unsupervised methods, including hierarchical clustering and principal components analysis (PCA), and supervised linear discriminant analysis (LDA). If the gene clusters are too large, top gene sets may be selected based on ANOVA and LDA to minimize overfitting. Patient samples may then be randomly divided into training and validation sets using 10-fold cross-validation and tested on multiple machine learning algorithms including, but not limited to, the ones stated above using scikit-learn (www.scikit-learn.org) and TensorFlow (www.tensorflow.org) deep learning environments. In some examples, samples may be randomly divided equally into groups; e.g., 10 groups, where one of 10 groups may be used as a validation set and the remaining 9 groups are used as training set. In such examples, the process may be repeated 9 more times with each group being used only once as validation set for a total of 10 iterations. Classification accuracy may be averaged across the 10 iterations. The probability that patients with more aggressive osteosarcoma (higher metastatic propensity) and with less aggressive osteosarcoma (lower metastatic propensity) are accurately classified by each algorithm then may be determined. Two to three algorithms achieving the highest average accuracy may be used for further studies. A common rate of success in the validation set may be used to define the operating true positive (sensitivity) and true negative (specificity) of the test using receiver operating characteristic (ROC) curves for each algorithm.

Rigor and Reproducibility: Sample size and power. Experimental applications of the techniques described herein may initially assess as many genes as may be feasible for the qNPA and qRT-PCR. Genes showing inconsistency in patient samples and/or those with little or no contributions to the sample classification then may be filtered out. As discussed above with respect to the data obtained from canines, biomarker sets may have a target percentage specificity and/or sensitivity, which may help enable prediction of rate of disease progression. Such biomarker sets may have species specificity.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various aspects of the disclosure have been described, These and other aspects are within the scope of the following claims.

What is claimed is:

1. A method for screening an organism for osteosarcoma, the method comprising:
isolating a plurality of exosomes from a sample of bodily fluid derived from the organism, wherein the plurality of exosomes comprises a plurality of molecules of ribonucleic acid (RNA);
determining respective RNA sequences for the plurality of molecules of RNA;
analyzing, by processing circuitry and using one or more machine learning models, expression level exhibited by the organism of each of a plurality of genes of a gene signature of osteosarcoma-linked genes based on the RNA sequences of the plurality of molecules of RNA occurring in the sample; and
determining, based on the analysis, a biological status of the organism.

2. The method of claim 1, wherein the plurality of genes of the gene signature comprises SKA, NEU1, PAF1, PSMG2, and NOB1.

3. The method of claim 1, wherein the plurality of genes of the gene signature comprises at least five genes selected from a group of osteosarcoma-linked genes.

4. The method of claim 1, wherein the plurality of genes are selected from a group of osteosarcoma-linked genes comprises the 25 genes.

5. The method of claim 1, wherein analyzing the expression level comprises applying the expression level of the plurality of genes of the gene signature to respective machine learning models of the one or more machine learning models.

6. The method of claim 5, wherein determining the biological status of the organism comprises determining that the machine learning models of the one or more machine learning models converge on the biological status from a plurality of biological statuses.

7. The method of claim 1, wherein the biological status of the subject comprises at least one of: a presence or absence of a disease state, a likelihood of development of a disease state, one or more characteristics of an existing disease state, a likelihood of a future progression of an existing disease state, or one or more characteristics of a predicted future progression of an existing disease state.

8. The method of claim 1, wherein the organism is a canine.

9. The method of claim 1, wherein the organism is a human.

10. The method of claim 1, further comprising:
determining that the biological status indicates a presence of a disease state for the subject; and
determining, based on the presence of the disease state, a treatment for the subject.

11. A method comprising:
obtaining a plurality of exosomes from each of a plurality of samples of bodily fluid derived from corresponding ones of a plurality of subjects, wherein one or more first subjects of the plurality of subjects have a biological status different from a biological status of one or more second subjects of the plurality of subjects, and wherein the plurality of exosomes from each of the plurality of samples of bodily fluid comprises a plurality of molecules of ribonucleic acid (RNA);
for each of the plurality of samples of bodily fluid:
determining, for substantially each molecule of the plurality of molecules of RNA, a corresponding RNA sequence; determining, for each corresponding RNA sequence, whether the RNA sequence is associated with exactly one corresponding gene sequence of a gene signature comprising a plurality of gene sequences;
determining an approximate number of times that each RNA sequence associated with exactly one corresponding gene of the gene signature occurs in the sample of bodily fluid; and
determining, using one or more machine learning models, a pattern of expression of the plurality of gene sequences of the gene signature associated with the sample of bodily fluid based on the approximate number of times that each RNA sequence associated with exactly one corresponding gene of the gene signature occurs in the sample of bodily fluid; and
associating, using the one or more machine learning models and for each subject of the plurality of subjects, the biological status of the subject with the corresponding pattern of expression of the of the plurality of gene sequences of the gene signature associated with the sample of bodily fluid from the subject.

12. The method of claim 11, wherein the biological status of the subject comprises at least one of: a presence or absence of a disease state, a likelihood of development of a disease state, one or more characteristics of an existing disease state, a likelihood of a future progression of an existing disease state, or one or more characteristics of a predicted future progression of an existing disease state.

13. The method of claim 11, wherein the gene signature comprises a plurality of genes comprising at least SKA, NEU1, PAF1, PSMG2, and NOB1.

14. The method of claim 11, wherein the gene signature comprises at least five genes selected from a group of osteosarcoma-linked genes.

15. The method of claim 11, wherein associating the biological status of the subject with the corresponding pattern of expression of the of the plurality of gene sequences comprises applying the pattern of expression to respective machine learning models of the one or more machine learning models.

16. The method of claim 15, wherein associating the biological status of the subject with the corresponding pattern of expression of the of the plurality of gene sequences comprises determining that the machine learning models of the one or more machine teaming models converge on the biological status from a plurality of biological statuses.

17. The method of claim 11, wherein the subject is a canine.

18. The method of claim 11, wherein the subject is a human.

19. A method comprising:
obtaining a plurality of exosomes from a sample of bodily fluid derived from an organism, wherein the plurality of exosomes comprises a plurality of molecules of RNA;
determining, for substantially each molecule of the plurality of molecules of RNA, a corresponding RNA sequence;
determining, for each corresponding RNA sequence, whether the RNA sequence is associated with exactly one corresponding gene sequence of a gene signature comprising a plurality of gene sequences;
determining an approximate number of times that each RNA sequence associated with exactly one corresponding gene of the gene signature occurs in the sample of bodily fluid;
analyzing, using one or more machine learning models, the approximate number of times that each RNA sequence associated with exactly one corresponding gene of the gene signature occurs in the sample of bodily fluid;
determining, using one or more machine learning models and based on the analysis, a pattern of expression exhibited by the organism of each the plurality of genes of the gene signature:
comparing, using one or more machine learning models, the pattern of gene expression to at least one known pattern of gene expression, wherein each of the at least one known patterns of gene expression is associated with a biological status; and
determining a biological status of the organism based on the comparison.

20. The method of claim 19, wherein the biological status of the organism comprises at least one of: a presence or absence of a disease state, a likelihood of development of a disease state, one or more characteristics of an existing disease state, a likelihood of a future progression of an existing disease state, or one or more characteristics of a predicted future progression of an existing disease state.
